(19) United States
(12) Patent Application Publication
(10) Pub. No.: US 2023/0372186 A1
(43) Pub. Date: Nov. 23, 2023

(54) WEARABLE COORDINATED AUTOMATIC TENSIONING APPARATUSES

(71) Applicant: Cincinnati Automation & Mechanics, LLC, Beavercreek, OH (US)

(72) Inventors: Roger Neil Rovekamp, JR., Kemah, TX (US); Keith Sawmiller, Beavercreek, OH (US)

(73) Assignee: Cincinnati Automation & Mechatronics,, LLC, Beavercreek, OH (US)

(21) Appl. No.: 18/030,299

(22) PCT Filed: Oct. 8, 2021
(86) PCT No.: PCT/US2021/054158

§ 371 Date: Apr. 5, 2023

Related U.S. Application Data
(60) Provisional application No. 63/089,764, filed on Oct. 9, 2020.

(51) Int. Cl. A61H 9/00

(52) U.S. Cl. CPC A61H 9/0092; A61H 2201/165; A61H 2230/045; A61H 2230/505; A61H 2201/5082; A61H 2201/5084; A61H 2201/5007

(57) ABSTRACT

A system for coordinating that actions taken by multiple apparatuses useful for automatically adjusting tension on a retention member and thereby applying pressure or other forces to an object. Sensors may be used to sense changes associated with the object involved or the environment, and an actuator may be included that automatically rotates a rotating member such as a gear or pulley to automatically adjust tension on the retention member. The adjustments in tension may be made performed automatically and many times per second based on control signals from control logic responsive to the sensors. Individual apparatuses may coordinate their activities according to input received from one or more sensors, and according to a control circuit, or multiple collaborating control circuits. The control circuit(s) may be configured to adapt automatically over time to optimize the overall behavior of the system.

### REFERENCE TO RELATED APPLICATION

The present application claims the benefit of United States Provisional Patent Application No. 63/089,764, filed Oct. 9, 2020 which is hereby incorporated by reference.

### BACKGROUND

The present disclosure relates to wearable devices for automatically controlling pressure or tension applied to an object that are configured to operate in concert. In some instances, the optimum pressure or tension applied to an object by any one wearable device may be dependent on movement of the object, movement of the wearable device relative to the object, environmental factors, or aspects of the state of the object itself, as well as actions taken by other similar wearable devices. In many cases, tension may be set manually such as by tightening a strap, tying a shoe lace, twisting a wire or cable until it is “tight”, applying a locking or crimped cable stay, and the like. Such systems cannot determine an optimum level of tension, particularly when multiple such manual devices are used together and/or cannot automatically adjust tension as needed. This can cause the tension level to be incorrectly set, thus damaging the tensioner itself, or damaging the object by either applying too much tension, or by not applying enough.

### SUMMARY

Disclosed is an automatic retention system for applying pressure or tension to one or more objects or limbs of a human or animal, the system optionally including multiple automatic retention apparatuses that optionally have a retention member surrounding a portion of at least one limb, and an actuator arranged and configured to engage the retention member. The actuator may be configured to actuate the retention member to adjust pressure applied to the object by the retention member. In another aspect, the system may include at least one sensor arranged and configured to sense changes in a sense parameter associated with the object, and at least one control circuit optionally responsive to the at least one sensor. The at least one control circuit may be configured to control the actuators of the multiple retention apparatuses to adjust pressure applied to the object, or objects, according to input received from the at least one sensor.

In another aspect, the system may include a garment configured to surround at least a portion of one of the one or more limbs. In another aspect, at least one of the multiple automatic retention apparatuses may be positioned outside a garment. In another aspect, at least one of the multiple automatic retention apparatuses is optionally positioned within a cavity defined within a garment. In another aspect, a retention member of at least one of the multiple automatic retention apparatuses is optionally woven into a garment.

In another aspect, a garment of the disclosed system optionally includes an actuator mount configured to couple an actuator of at least one of the multiple automatic retention apparatuses adjacent to a retention member. In another aspect, a retention member of at least one of the multiple automatic retention apparatuses optionally includes a cloth strap that may be mounted to the garment at a predetermined mount location. In another aspect, a cloth strap may be mounted inside a garment, and the garment may define an opening through which the actuator engages the retention member to adjust tension on the cloth strap.

In another aspect, a garment of the disclosed system may define an interior passageway and at least a portion of one retention member of the multiple automatic retention apparatuses may be positioned inside the interior passageway. In another aspect, and actuator of the at least one retention member is optionally mounted outside an interior passageway of the garment. In another aspect, a garment optionally includes multiple mounts generally corresponding to each of the multiple automatic retention apparatuses that may be mounted to the garment.

In another aspect, at least one automatic retention apparatus of the multiple automatic retention apparatuses may be mounted remote from the at least one control circuit. In another aspect, at least one automatic retention apparatus of the multiple automatic retention apparatuses may be mounted to a housing and the at least one control circuit is optionally mounted inside the housing.

In another aspect, an upper retention apparatus of the at least one automatic retention apparatuses may be mounted at an upstream location of a limb of the one or more limbs, and a separate lower retention apparatus of the at least one automatic retention apparatuses may be mounted at a downstream location of the limb. In another aspect, the upstream and downstream portions of a limb are coupled together by a joint of the limb.

In another aspect, system optionally includes a frame that may be configured to receive at least a portion of a limb of the one or more limbs, and at least one of the multiple retention members may be mounted to the frame. In another aspect, a frame may be included that has two frame members on opposing sides of a limb, and the two frame members may be longitudinally aligned with a reference plane.

In another aspect, the system may also include multiple support elements coupled together and coupled to a frame, the multiple support elements may be aligned with a reference plane. The multiple support elements and the frame may be coupled together and rotatable substantially parallel to the reference plane, and are optionally prevented from rotating away from the reference plane by virtue of multiple projecting members positioned within corresponding cavities of the support elements.

In another aspect a frame is included that comprises an upper portion optionally mounted to an upper part of a limb, and a lower portion mounted to a lower part of the limb. The upper and lower parts of the limb are optionally coupled together by a joint, at least one upper retention member of a retention apparatus may be mounted to the upper portion to surround at least a portion of the upper part of the limb. At least one other retention member of another separate retention apparatus may be mounted to the lower portion and may surround at least a portion of the lower part of the limb.

In another aspect, at least one control circuit optionally includes multiple individual control circuits. The multiple automatic retention apparatuses may be separately responsive to one or more separate control circuits of the multiple individual control circuits. In another aspect, separate control circuits may be in communication with and responsive to at least one other control circuit of the multiple individual control circuits.

In another aspect, the at least one sensor mentioned above optionally includes multiple individual sensors. The multiple individual control circuits may be separately responsive to one or more sensors of the multiple individual sensors. In another aspect, the at least one sensor optionally includes, but is not limited to, a blood pressure sensor, a temperature sensor configured to determine a temperature of the limb, a heart rate sensor, a temperature sensor configured to determine the ambient temperature around the limb, an accelerometer, an Inertial Measurement Unit (IMU), or sensors that measure oxygenation, respiratory rate, perspiration, brain function at a brain-machine interface, make cognitive function assessments, or any combination thereof.

In another aspect, the at least one control circuit may be a single control circuit operatively coupled to some or all of the multiple automatic retention apparatuses. In another aspect, the at least one sensor optionally includes a first sensor that may be mounted to a retention member of the multiple automatic retention apparatuses. This first sensor may be configured to generate input based on tension in the retention member. A second sensor may be mounted to an object the retention member at least partially surrounds, and the second sensor may be configured to generate input based on movement of the object.

In another aspect, the system optionally includes a cable inside a conduit, wherein the cable is coupled adjacent a first end of an actuator of at least one of the multiple automatic retention apparatuses. A cable actuator may be included that is responsive to a control circuit is optionally coupled to the cable adjacent a second end. The cable may be selectively movable within the conduit according to movement of the cable actuator, and the actuator may be configured to adjust tension applied by the retention member according to movement of the cable relative to the conduit. In another aspect, the system optionally includes a cable actuator that has an electric motor mechanically coupled to a rotating member. The electric motor may be responsive to control input from the at least one control circuit, and the at least one control circuit may be programmed to control the electric motor to rotate in a first direction and a second direction opposite the first direction to adjust the position of the cable relative to the conduit. In another aspect, the system optionally includes a conduit that may be anchored at a first end to a first cable mount of the at least one automatic retention apparatus, and the conduit may be anchored at a second end to a cable second mount of a cable actuator. In another aspect, the system also optionally includes a garment configured to surround at least a portion of one of the one or more limbs, and the garment may define an interior passageway capable of receiving a cable and conduit positioned inside the interior passageway. In another aspect, the automatic retention system optionally includes multiple cables separately positioned inside individual conduits. The multiple cables may extend between separate actuators of the multiple automatic retention apparatuses and the at least one control circuit. Multiple cable actuators may be included that are responsive to the control circuit and are each individually coupled at a first end of each of the multiple cables. The multiple cables may be selectively movable within the individual conduits according to movement of cable actuators, and the separate actuators may be coupled to a second end of each of the multiple cables. The separate actuators are optionally configured to adjust tension applied by corresponding retention members according to movement of the individual cables relative to the individual conduits.

In another aspect, the actuator of at least one of the multiple automatic retention apparatuses optionally includes a rotating member with multiple teeth preferably engaging one or more recesses defined by the retention member. In another aspect, the rotating member may be rotatable in a first direction to reduce tension of the retention member, and the rotating member may be rotatable in a second direction opposite the first direction to increase tension of the retention member. The actuator may be mechanically coupled to the rotating member, and the actuator may be arranged and configured to rotate the rotating member in the first direction and the second directions to increase or decrease tension of the retention member in response to input from the control circuit.

In another aspect, the retention member is optionally an elongate retention member, and one or more projections and recesses may be defined in a portion of the retention member that is wider than it is thick, and narrower than it is long. In another aspect, at least one of the one or more recess and projections may include through holes interspersed along the retention member. In another aspect, a rotating member may be included that rotates around an axis of rotation that may be substantially parallel to a longitudinal axis defined by the retention member. In another aspect, a rotating member may rotate around an axis of rotation that is substantially perpendicular to a longitudinal axis defined by the retention member. In another aspect, rotating a rotating member in a first direction optionally displaces a first portion of the retention member relative to a second portion of the retention member to adjust tension of the retention member. In another aspect, the actuator of at least one of the multiple automatic retention apparatuses may include an electric motor mechanically coupled to a rotating member. The electric motor may be responsive to control input from the at least one control circuit, wherein the at least one control circuit is programmed to control the electric motor to rotate in the first and second direction to adjust tension of the retention member. In another aspect, the actuator of at least one of the multiple automatic retention apparatuses optionally includes a rotating member engaging with at least a portion of the retention member. The rotating member may be rotatable in a first direction to reduce tension of the retention member, and it may be rotatable in a second direction opposite the first direction to optionally increase tension of the retention member. The actuator may be mechanically coupled to the rotating member, and the actuator may be arranged and configured to rotate the rotating member in the first direction and the second directions to optionally increase or decrease tension of the retention member in response to input from the control circuit. In another aspect a portion of the retention member of the present disclosure may engage the rotating member and it may be arranged and configured to wrap around the rotating member as it rotates.

In another aspect, a control circuit of the at least one control circuits may be configured to control the actuators according to values for one or more operating parameters, and criteria for one or more rules. In another aspect a control circuit of the present disclosure is optionally configured to receive one or more values for operating parameters from a remote computing device via a communications link. In another aspect, the control circuit may be configured to receive criteria for one or more rules from a remote computing device via a communications link.

In another aspect, the control circuit is optionally configured to automatically determine and update at least one value for one or more operating parameters and at least one criteria for one or more rules. In another aspect, the control circuit optionally includes a memory, and the control circuit may be configured to retain an operational history of operating parameters including a first value of an operating parameter retained in the memory at a first time, and a second value of the operating parameter retained in the memory at a second later time. The first and second values may be used (possibly along with other values) to determine a third new value for the operating parameter. In another aspect, operating parameters, and/or an operational history of an automatic retention apparatus may be sent to a remote computer via a communication link.

In another aspect, the actuator of at least one of the multiple automatic retention apparatuses optionally includes a manual control that may be mechanically coupled to a rotating member. The manual control may be arranged and configured to rotate a rotating member of the automatic retention apparatus in a first and second direction to adjust tension of the retention member based on input from a user.

In another aspect, the system may include one or more inflatable cavities positioned between a retention member of an automatic retention apparatus and the at least one limb. The actuator is optionally arranged and configured to inflate the one or more inflatable cavities to adjust pressure applied to the at least one limb by the retention member.

In another aspect, the system may include multiple automatic retention apparatuses that include a retention member optionally surrounding a portion of at least one limb of the one or more limbs. One or more inflatable cavities may be positioned between the retention member and the at least one limb, and an actuator is optionally arranged and configured to inflate the one or more inflatable cavities to adjust pressure applied to the at least one limb by the retention member. At least one sensor may be arranged and configured to sense changes in a sense parameter associated with the one or more limbs, and at least one control circuit is optionally responsive to the at least one sensor, and the at least one control circuit may be configured to control the actuators of the multiple retention apparatuses to adjust inflation of the one or more inflatable cavities according to input received from the at least one sensor.

In another aspect, an automatic retention apparatus optionally includes one or more inflatable cavities such as first and second cavities of the one or more inflatable cavities that are separate and distinct, and the actuator may be configured to selectively inflate the first cavity at a first pressure, and the second cavity at a second pressure that is different than the first pressure. In another aspect, the system optionally includes an automatic retention apparatus with one or more inflatable cavities that are in fluid communication with each other. In another aspect, the system includes an automatic retention apparatus with one or more inflatable cavities that may be arranged circumferentially around the retention member. In another aspect, the system also including a fluid compression apparatus in fluid communication with one or more of inflatable cavities of an automatic retention apparatus.

The fluid compression apparatus may be arranged and configured to introduce a fluid into the cavities to inflate the one or more inflatable cavities, or optionally to extract fluid from the cavities to deflate one or more cavities. In another aspect, the system optionally includes a fluid compression apparatus that may include an air compressor responsive to the at least one control circuit, and the fluid used to inflate the cavities is air. In another aspect, the fluid compression apparatus optionally includes a squeeze bulb. In another aspect, the a one way valve may be included that is optionally configured to open when the compression apparatus introduces fluid into the cavities, and to otherwise automatically close to retain the fluid in the cavities. In another aspect, the system optionally includes a first sensor mounted to a retention member of the multiple automatic retention apparatus that is configured to optionally generate input based on an interior pressure of the fluid in at least one inflatable cavity. A second sensor may also be included and may be mounted to an object the retention member at least partially surrounds.

Further forms, objects, features, aspects, benefits, advantages, and examples of the present disclosure will become apparent from a detailed description and drawings provided herewith.

### BRIEF DESCRIPTION OF THE DRAWINGS

FIG. 1 is a component diagram illustrating one example of components that may be included in an automatic retention apparatus of the present disclosure.

FIG. 2 is a component diagram illustrating aspects of an automatic retention apparatus of the present disclosure.

FIG. 3 is a component diagram illustrating alternative configurations for an automatic retention apparatus of the present disclosure.

FIG. 4-5 are component diagrams illustrating aspects of the placement of automatic retention apparatuses of the present disclosure.

FIG. 6 is a partial cutaway view of a garment illustrating additional aspects of the mounting and placement of automatic retention apparatuses of the present disclosure.

FIG. 7 is a partial perspective view of a piece of fabric illustrating additional aspects of the mounting and placement of automatic retention apparatuses of the present disclosure.

FIG. 8 is a partial perspective view of a garment illustrating additional aspects of the mounting and placement of automatic retention apparatuses of the present disclosure.

FIG. 9 is front view of a garment, or garments, illustrating mounting and placement of automatic retention apparatuses of the present disclosure.

FIG. 10 is a component diagram illustrating communication aspects for automatic retention apparatuses of the present disclosure.

FIG. 11 is cutaway component diagram illustrating alternative configurations for an automatic retention apparatus of the present disclosure.

FIG. 12 is a component diagram illustrating additional communication aspects for automatic retention apparatuses of the present disclosure.

FIG. 13 is a component diagram illustrating aspects of the placement and operation of automatic retention apparatuses of the present disclosure.

FIG. 14 is a component diagram illustrating a frame system for mounting automatic retention apparatuses of the present disclosure.

FIG. 15 is a partial cutaway view illustrating other aspects of a frame system for mounting automatic retention apparatuses of the present disclosure.

FIG. 16 is a perspective view illustrating additional aspects of a frame system for mounting automatic retention apparatuses of the present disclosure.

FIG. 17 is a component diagram illustrating additional aspects of a frame system for mounting automatic retention apparatuses of the present disclosure.

FIG. 18 is a component view of sensor placement for use with an automatic retention system of the present disclosure.

FIG. 19 is a partial cutaway view and component diagram of an actuation mechanism for use with the automatic retention apparatuses of the present disclosure.

FIG. 20 is another partial cutaway view and component diagram illustrating aspects of an actuation mechanism for use with the automatic retention apparatuses of the present disclosure.

FIG. 21 is a component diagram illustrating placement of the actuation mechanisms of FIGS. 19 and 20 in a garment according to the present disclosure.

FIG. 22 is cutaway component diagram illustrating another alternative configurations for an automatic retention apparatus of the present disclosure.

FIG. 23 is perspective view and component diagram illustrating another configuration for an automatic retention apparatus of the present disclosure.

FIG. 24 is cutaway component diagram illustrating another alternative configurations for an automatic retention apparatus of the present disclosure.

FIG. 25-28 are cross-sectional diagrams illustrating alternative configurations for an automatic retention apparatus of the present disclosure.

FIG. 29 is a component flow diagram illustrating one example of a control circuit for controlling the automatic retention apparatus of the present disclosure.

### DETAILED DESCRIPTION

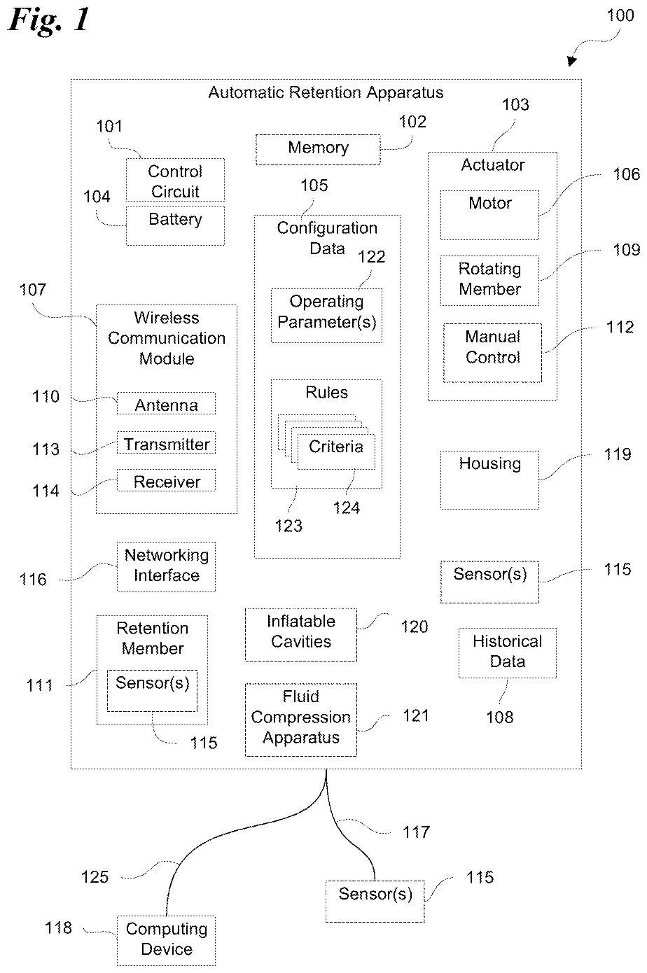

FIGS. 1-4 illustrate examples of components that may be included in the disclosed automatic retention system for coordinating the application of pressure to one or more limbs. The various components disclosed throughout this disclosure, and the configurations presented in the figures, may be common to any or all of the disclosed examples of an automatic retention system. In general, the disclosed automatic retention apparatuses apply pressure by applying tension to a retention member such that by applying tension, the retention member tightens around the object. This configuration is provided as merely an example is any suitable method of applying pressure may be used.

An automatic retention system of the present disclosure may include multiple automatic retention apparatuses with components like those illustrated in FIG. 1. For example, an automatic retention apparatus 100 may include a control circuit 101 for processing data and generating commands or instructions to other components in the disclosed system to control the operating behavior of one such device, or of multiple coordinating devices working together. Control circuit 101 may include a processor, logic circuits, digital or analog circuitry, or any combination thereof. The control circuit 101 may be useful for accepting input and generating output for controlling operational characteristics of the apparatus 100, or of multiple such apparatuses working together as part of the overall system. A battery 104 may be included for providing power to the control circuit 101, and to other components of the apparatus that may accept electrical power.

A memory 102 may be included for storing information such as configuration data 105 and historical data 108. In another aspect, memory 102 may be configured to store data about the operation of the automatic retention apparatus 100, or of other cooperating apparatuses. Examples of this historical data include, but are not limited to, the extent of tension applied by the device, the extent of tension applied by other devices, the time when the device was activated to apply or release tension, the sensor input that triggered the changes in tension, or the control logic that was triggered to cause the change in tension, or any combination thereof.

Data obtained over time from sensor input may also be stored in memory 102 for processing by control circuit 101, or for processing by other computing devices which may analyze the data to change configuration data 105 to improve the overall performance of the apparatus 100. Thus memory 102 may also be configured to store data values representing sense parameters detected by sensors 115 that was provided by the sensors as input to the control circuit 101. Historical data 108 may include dates, times, locations, or other metadata. Configuration data 105 may include parameter values for configuring the operation of the disclosed automatic retention apparatuses. Such configuration data may include values for one or more operating parameters, and optionally, criteria for one or more rules.

A wireless communication module 107 may be included and may include an antenna 110, transmitter 113, and receiver 114. The antenna may be used by the transmitter and receiver to send and receive wireless communications to a computing device 118 to, for example, send and receive updated configuration data, historical data, and/or control signals. Antenna 110 may be configured to resonate according to radio waves carrying signals defining data sent and received by wireless communication module 107. Transmitter 113 may use antenna 110 to send signals, and receiver 114 may also use antenna 110 to receive signals defining data to be processed by control circuit 101 and/or stored in memory 102. Signals sent and received by the transmitter and receiver may be sent via any suitable medium such as via radio waves, by modulating visible or invisible light, and the like.

A network interface 116 may be included and may implement various communication protocols useful for interacting with remote devices over a communications link that may be connected to a network such as the Internet. Such a communications link may be a wireless communications link implemented using wireless communication module 107, or a physical communication link implemented using wires, optical fibers, and the like. For example, wireless communication module 107 may transmit and receive signals which may then be processed according to the protocols recognized by network interface 116 in order to implement a communications link.

Retention apparatus 100 may include a retention member 111 for applying tension to an object. One or more sensors 115 may be included in or coupled to retention member 11. Sensors 115 may optionally be included in, or mounted to, retention member 111, or included in, or mounted to, automatic retention apparatus 100. In another aspect, sensors 115 may include sensors arranged and configured to sense changes in a sense parameter associated with an object retained by the device 100. Retention apparatus 100 optionally defines and/or includes one or more inflatable cavities 120 which are optionally positioned between the retention member 111 and the at least one limb or other object the retention apparatus is coupled to. In another aspect, the actuator 103 is optionally arranged and configured to inflate the one or more inflatable cavities 120 to adjust pressure applied to one or more limbs by the retention member.

In another aspect, the automatic retention apparatus 100 optionally includes a fluid compression apparatus 121 in fluid communication with one or more of the inflatable cavities 120. The fluid compression apparatus 121 may be arranged and configured to introduce a fluid into the cavities to inflate them (if the apparatus is equipped with such inflatable cavities).

In another aspect, sensors 115 may include environmental sensors arranged and configured to sense changes in an environmental sense parameter associated with an environment surrounding the sensor. These environmental sensors may be positioned in the housing 119, or elsewhere outside the housing. The control circuit 101 may be responsive to the environmental sense parameter as these parameters may represent any suitable environmental aspect such as speed, angular momentum, velocity, movement, acceleration, temperature, altitude, attitude (that is, angle of inclination with respect to earth), or any combination thereof. Sensors 115 may be mounted to other objects interacting with apparatus 100 such as in the case of wired or wireless sensors sending data received as signals via a communication link.

An actuator 103 may be included and configured to act on retention member 111 to increase or decrease tension on the retention member to vary the resulting tension or pressure on any objects to be held in place by retention member 111. A motor 106 may be included in actuator 103 and coupled to a rotating member 109 such as the gear, can, pulley, and the like, or any combination thereof. An optional manual control 112 may be coupled to rotating member 109 to manually adjust the tension on retention member 111 by manually adjusting rotating member 109. This manual rotation may be performed along with, or as an alternative to, the automatic rotation provided by motor 106.

In another aspect, control circuit 101 may be responsive to input from at least one sensor of sensors 115 and may be configured to control the actuator accordingly. A control circuit of the present disclosure may control the disclosed actuator to energize the motor to rotate the rotating member in the first or second direction to automatically adjust tension on a limb or another object. For example, control circuit 101 may control the actuator to rotate in the first and second directions with the direction and number of rotations being a function of the input received from sensor 115. In another aspect, some or all of the components of 100 may be mounted to, or mounted inside, a housing 119. In another aspect, the control circuit 101 may be configured to increase tension on the retention member when the sense parameter matches a first target criteria, and/or to decrease tension on the retention member when the sense parameter matches a second target criteria. This first target criteria, second target criteria, the configuration for which direction to rotate the rotating member, the sensor input to consider in making the determination, and other related rules or criteria are examples of configuration data 105 that may be maintained by the automatic retention apparatus 100.

In another aspect, control circuit 101 may be configured to control actuator 103 according to values for one or more operating parameters 122, and criteria 124 for one or more rules 123. In another aspect, the control circuit 101 is optionally configured to receive one or more values for the operating parameters 122, and/or criteria 124 for the rules 123, from a remote computing device 118 via a communication link 125. This communication link may be wired, wireless, or of any other suitable type transferring operating parameters 122.

In another aspect, the control circuit 101 may be configured to automatically determine and update at least one of the one or more values for one or more operating parameters 122, and/or at least one of the criteria 124 of the rules 123. Control circuit 101 may include artificial intelligence, neural networks, deep learning algorithms, or other similar algorithms for automatically adjusting the behavior of the automatic retention apparatus based on past experience and current sensory inputs. These algorithms may be optimized, or tuned, to achieve optimal results given the limited computational resources that may be available to automatic tension apparatus 100.

In one example, control circuit 101 optionally includes, or has access to, memory 102. The control circuit may be configured to retain an operational history of operating parameters including a first value of an operating parameter retained in the memory at a first time, and a second value of the operating parameter retained in the memory at a second later time, and wherein the first and second values are used to determine a third new value for the operating parameter. In another aspect, the operational history and historical data 108 may be sent to a remote computing device 118 via the communication link 125.

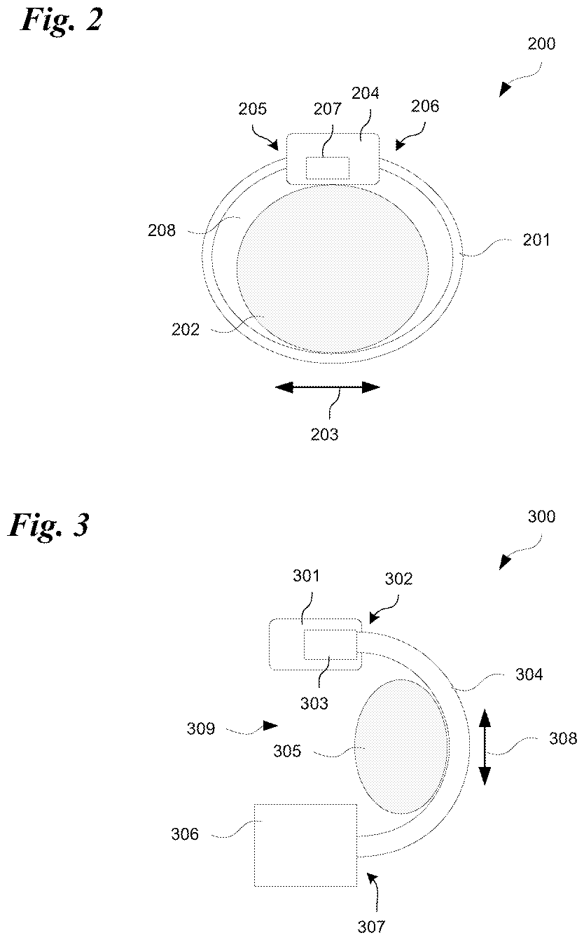

FIG. 2 illustrates additional aspects common to the disclosed automatic retention apparatuses as shown at 200. A retention member 201 is shown that is separate and distinct from an object 202 (such as a limb, or portion thereof). Retention member 201 is optionally positionable around the object 202, and the retention member arranged 201 is optionally arranged and configured to engage the object to increase or decrease tension on the object. In this example, retention number 201 may be rigid, or semi-rigid and therefore may be movable in the direction of 203. In this respect, retention member 201 may operate to apply tension by pulling toward the object 202, or alternatively by pushing toward the object 202. In another aspect, 201 may be flexible, or semi-flexible, or a fabric, cable, or wire have multiple strands, or a single strand. In this configuration, retention member 201 is optionally only able to apply tension to the object by pulling toward the object 202.

In another aspect, retention member 201 may be mounted to a housing 204. In one example, retention member 201 may be mounted to housing 204 at, or adjacent to, a first end 205, a second end 206, or any combination thereof. In another aspect, the retention member 201 may engage an actuator 207 at, or adjacent to, the first or second ends 205 and 206. In another aspect, actuator 207 may engage retention member 201 at any point along its length.

In another aspect, the retention member 201 and the housing 204 together define an opening or empty space 208 within which object 202 may be positioned. For example, object 202 may be positioned such that retention member 201 is substantially perpendicular to the portion of object 202 that is positioned within the opening 208. However, any suitable orientation of object 202 with respect to the retention member 201 may be used.

In one nonlimiting example, object 202 may be a human or animal appendage (such as an arm, leg, wrist, and the like), and retention member 201 may operate to automatically increase or decrease tension applied to the human or animal appendage. For example, retention apparatus 200 may automatically adjust tension of retention member 201 to increase or decrease pressure applied to the human or animal appendage, such as in the case of controlling a flow of blood or other bodily fluids at or adjacent to a wound.

Another aspect of the automatic retention apparatus of the present disclosure is illustrated at 300 in FIG. 3. A retention member 304 is shown that is separate and distinct from an object 305. Retention member 304 is optionally positionable around an object 305, and the retention member arranged 304 is optionally arranged and configured to engage the object to increase or decrease tension on the object. In this example, retention member 304 may be rigid, or semi-rigid and therefore may be movable in the direction of 308 depending on actuator 303. In this respect, retention member 304 may operate to apply tension by pulling toward the object 305, or alternatively by pushing toward the object 305.

In another aspect, retention member 304 may be optionally mounted to an anchoring object 306 at, or adjacent to, a first end 307. In one example, the anchoring object 306 is a brace, frame, or other supporting structure. In another example, the anchoring object 306 is a garment, or a portion thereof. In that example, a first in 307 may be coupled to fabric of a garment such as by an adhesive, by being woven into the fabric, stitched to the fabric, or otherwise coupled by means of a coupling device such as a snap, clasp, hook and loop material, button, zipper, or any other suitable coupling device.

A second end 302 may engage actuator 303 within a housing 301 of the automatic retention apparatus. In this example, retention member 304 may be configured to apply tension to the object 305 where one end of the retention member 304 is acted on by an actuator 303, and the other end is mounted to an anchor that is separate from the housing 301.

In another aspect, the retention member 309, anchoring object 306, and the housing 301 together define an opening 309 within which the object 305 may be positioned. For example, object 305 may be positioned such that retention member 304 is substantially perpendicular to the portion of object 305 that is positioned within the opening 309.

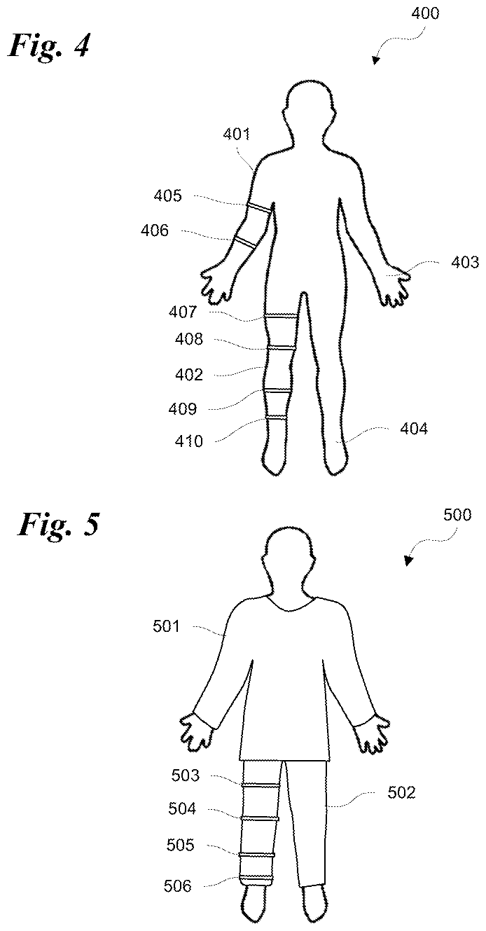

In one aspect illustrated in FIG. 4, the retention members of one or more automatic retention apparatuses of the present disclosure optionally surround a portion of at least one limb such as, 401, and/or 402. Limb 401 is, in this case an arm of a human being, and is one of one or more limbs that includes, but is not limited to, the arm 401, a leg 402, a hand 403, and a foot 404. In another example, the retention members 407, 408, 409, and 410 are optionally included and are arranged and configured to surround the limb 402. As disclosed herein, the retention members 405-410 may be any of the present disclosure, and may be coupled to individual control and actuation mechanisms that allow them to work in concert together, or individually.

In another aspect, an actuator 103 of the present disclosure may be arranged and configured to engage the retention member 11l (or any others such as 405-410). The actuator 103 may be configured to actuate the retention member 111 to adjust tension applied to the limbs by the retention members of the present disclosure. In another aspect, the sensor(s) 115 may be arranged and configured to sense changes in a sense parameter associated with the one or more limbs of the present disclosure such as limbs 401-404. This input from multiple sensors in multiple locations around the limbs may be used by the automatic retention apparatuses to coordinate a response.

In another aspect illustrated in FIG. 5 at 500, the disclosed automatic retention system may include a garment 501 and/or 502 which are optionally configured to surround at least a portion of one or more limbs of an animal or a human wearing the garment. In another aspect, 501 and 502 may be considered different parts of a single garment. For example, 501 may be a coat, shirt, blouse, smock, or other garment while 502 may be a pair of pants, jeans, or other similar garment. In another example, 501, and 502 may together be thought of as a single garment such as in the case of a uniform that might be worn by a member of the military, a first responder, nurse or doctor, to name a few nonlimiting examples.

In another aspect, the multiple retention apparatuses of the present disclosure may be mounted to a garment, either inside the garment, outside the garment, or any combination thereof. For example, at least one of the disclosed multiple automatic retention apparatuses may be positioned outside the garment at 503. Other retention apparatuses may be included as well such as 504, 505, and 506. No particular limitation on the number or placement of retention apparatuses is implied in the figures included herein. Rather, the figures provided are exemplary only and not restrictive.

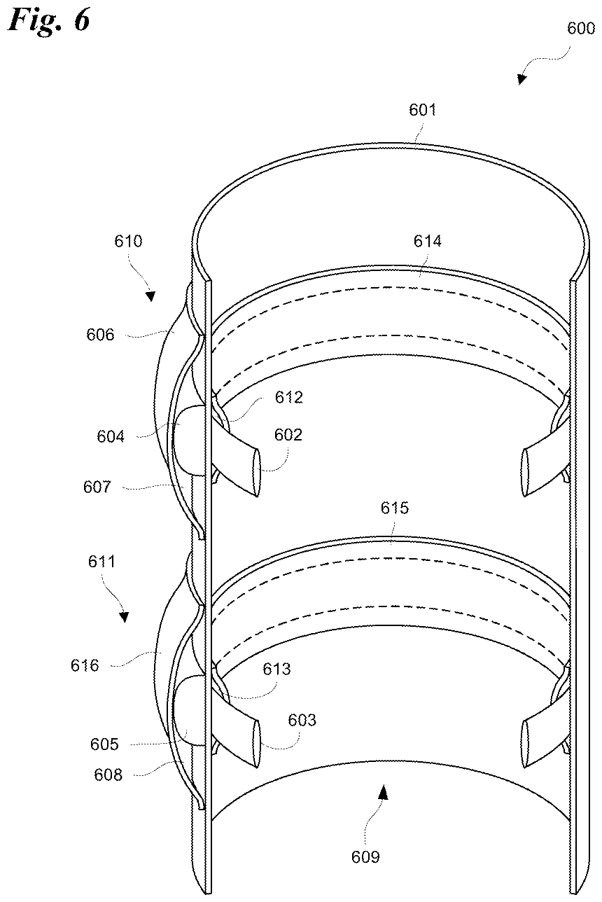

In another aspect illustrated in FIG. 6 at 600, a garment of the present disclosure defines an interior passageway and at least one retention member of the multiple automatic retention apparatuses is positioned inside the interior passageway. A garment 601 defines a passageway 609 through which an object or a portion of a limb may pass. Examples include a sleeve, pant leg, or a waist or torso area of a garment, or other passageway. In this example, two separate automatic retention apparatuses 610, and 611 are optionally mounted to the garment 601. Any suitable number of retention apparatuses of the present disclosure may be mounted in this fashion along an interior passageway of the garment.

At 610, a retention member 602 may be mounted in the garment within a cavity 612 defined by the garment. In this example, retention member 602 is optionally mounted on an inside surface of 609 and extends annularly around the inside of the passageway. In this example, a portion of the garment 614 is coupled to the garment to define passageway 612. The garment portion 614 may be coupled to the inside surface of passageway 609 by any suitable method such as by an adhesive, by stitching, by zippers, snaps, or by a hook and loop fastener arrangement, to name a few nonlimiting examples. Housing 604 may be held in position to engage retention member 602 by any suitable means.

In another aspect, the portion of the garment 614 and/or 615 may operate to reduce frictional forces caused by retention member 602 and/or 603 as they are tightened or loosened. As tension is applied to the disclosed retention members by an actuator, the retention members of the present disclosure may slide across the surface of the limb or other object to which the retention member is applied. These frictional forces may increase to the point of damage to the limb or object, or to the point of sever discomfort for the person wearing the retention apparatus. A sleeve or sheath like what is shown at 614 and 615 may be useful for reducing or eliminating the friction applied to the object by the retention member. In this example, the retention member moves relative to the interior surface of the sleeve or sheath, and not relative to the surface of the limb. A sleeve, sheath, or other protective layer may be advantageously applied to any of the disclosed examples of a retention member.

In another aspect, an additional layer of material having a lower coefficient of friction than the retention member material may be applied to the retention member itself. This friction reducing layer may be applied by any suitable means such as by way of a spray on coating, by an adhesive between the layer and the retention member, by altering the chemical properties of the surface of the retention member itself to infuse it with a lower friction compound or material, or any other suitable means, or combination thereof.

A housing 604 of an automatic retention apparatus of the present disclosure may be mounted to an outside surface of the cavity 609 and may be maintained in position relative to retention member 602 by another portion 606 of the garment. 606 may be configured to define a cavity 607 which may be of suitable dimensions to partially, or completely, enclose housing 604. For example, 606 may include a pocket with a zipper, snap, button, or hook and loop system that is provided as part of the garment. This configuration may allow the automatic retention apparatuses of the present disclosure to be placed in the garment after manufacturing of the garment is complete. 606 may also be selectively available for opening and closing in order to access aspects of the automatic retention apparatus for maintenance or replacement as needed. For example, 606 may be selectively opened and closed in order to remove the electronics and actuating portions of the automatic retention apparatus that are in housing 604 so that the garment 601 may be laundered or otherwise cleaned. In another aspect, the automatic retention apparatuses may be considered disposable and thus 606 and 614 may be configured without selective opening and closing features.

The housing 604 may include other aspects of an automatic retention apparatus of the present disclosure such as an actuator, rotating member, sensors, control circuit, and the like. Any of the components illustrated in FIG. 1 may be included, as well as others disclosed herein. Thus it is illustrated in FIG. 6 that a garment may be configured so that at least one of the multiple automatic retention apparatuses is positioned within a cavity defined within the garment.

A garment may be configured as illustrated herein to include multiple automatic retention apparatuses as shown at 600. At 611, a configuration of an automatic retention apparatus shown that is similar to what is shown at 610. A housing 605 containing components such as those illustrated in FIG. 1 is maintained in a cavity 608 defined by garment 601 including separate portions of the garment 66, and 615. Housing 605 may be thus held in position to engage retention member 603 according to any of the examples of the present disclosure.

As illustrated in FIG. 6, multiple automatic retention apparatuses may be incorporated into a garment of the present disclosure so as to apply tension to a limb, or other appendage of a human or animal.

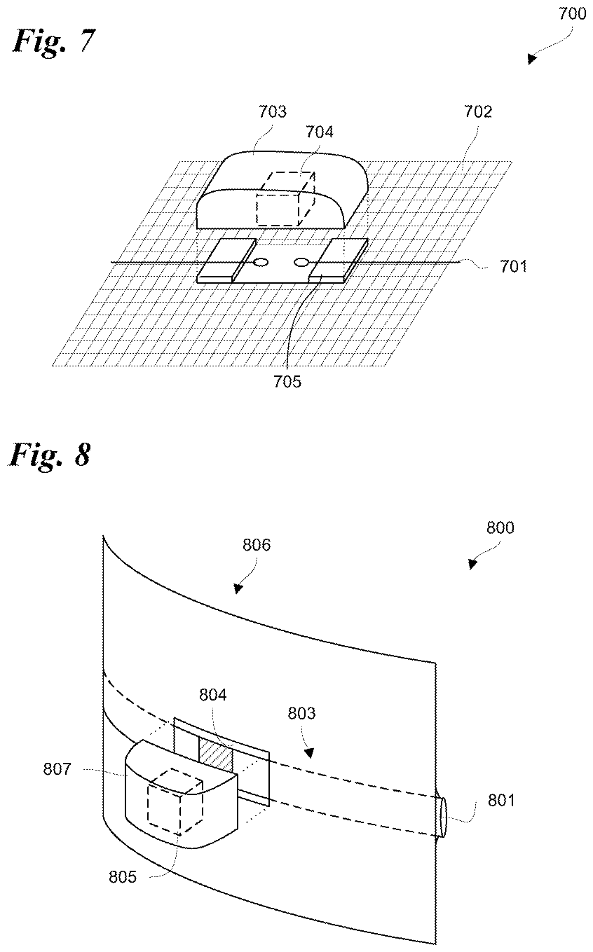

In another aspect illustrated in FIG. 7 at the 700, a retention member 701 of at least one of the multiple automatic retention apparatuses of the present disclosure is optionally woven into the garment 702. This example, an automatic retention apparatus includes a housing 703 that includes an actuator 704. The garment 702 optionally includes an actuator mount 705 configured to couple the housing 703 and actuator 704 of at least one of the multiple automatic retention apparatuses adjacent to the retention member 701. In this position, actuator 704 is positioned to engage retention member 701 to adjust the tension on retention member 701 as disclosed herein.

In another aspect illustrated at 800 in FIG. 8, a retention member 801 of at least one of the multiple automatic retention apparatuses 807 of the present disclosure optionally includes a cloth strap that is mounted to the garment 802 at a predetermined mount location 803. The cloth strap is optionally mounted inside the garment 802. The garment may define an opening 804 through which an actuator 805 may engage the retention member 801 to adjust tension on the cloth strap. In another aspect, the actuator 805 of an automatic retention apparatus 807 is mounted outside an interior passageway 806 that may be defined by the garment 802. In this example, the limb, or object to which pressure is to be applied is optionally arranged on the inside of an interior passageway at 806, similar to the passageway 609 of FIG. 600. The automatic retention apparatus is here mounted outside the garment to provide for ease of maintenance, replacement, or inspection. This also allows for the portion of the automatic retention apparatus 807 that includes the electronics, motors, control logic, and other components that may be susceptible to environmental damage to be removed as needed to avoid such damage.

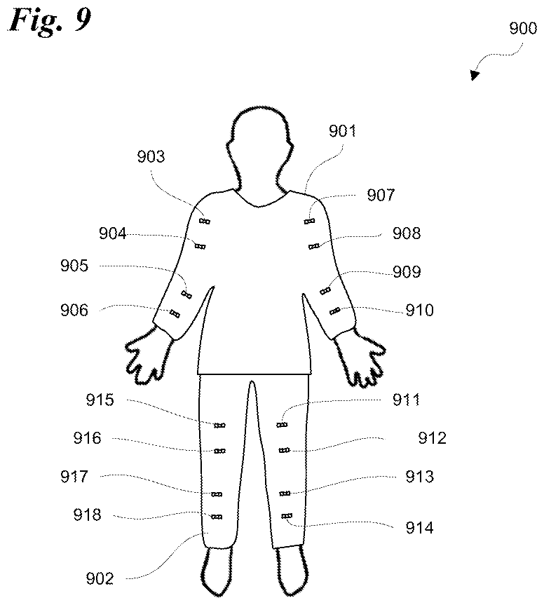

In another aspect illustrated in FIG. 9, a garment 900 that optionally includes multiple mounts, or mount points, that optionally correspond to one or more of the multiple automatic retention apparatuses that may be mounted to the garment. For example, mounts 903 and 904, and mounts 907 and 909, may be positioned on sleeves of the garment 901. In this configuration, the disclosed automatic retention apparatuses may be positioned on the garment in an advantageous location to partially or completely surround a limb such as the upper arms. Mounts 903, 904 and 907, 909 thus allow the disclosed automatic retention apparatuses to optionally apply external pressure to the left and/or right humerus, biceps, triceps, and tissue, nerves or blood vessels that are nearby.

Similarly, mounts 906, 905, 909, and 910 optionally provide positioning for the disclosed automatic retention apparatuses adjacent to the lower arms. This configuration allows for pressure to be applied to the lower arm structures including the radius, ulna, and the nerves, blood vessels, and tissue nearby. In another aspect, mounts 915-918, and 911-914 similarly provide mount points for the disclosed retention system to apply pressure to other limbs such as the upper and lower leg regions. In another example, automatic retention apparatuses may be mounted at all of the mount points 903-914 thus providing the opportunity for multiple apparatuses to surround a portion of multiple limb areas to apply external pressure to muscles, bones, blood vessels, tissue, or other aspects as needed. For example, wounds to the tissue, bones, or other anatomical structures may be automatically responded to quickly and effectively to reduce or eliminate loss of blood, to stabilize damaged or broken bones or joints, and/or to automatically apply a tourniquet in the case of catastrophic injuries where extreme lifesaving measures may be necessary.

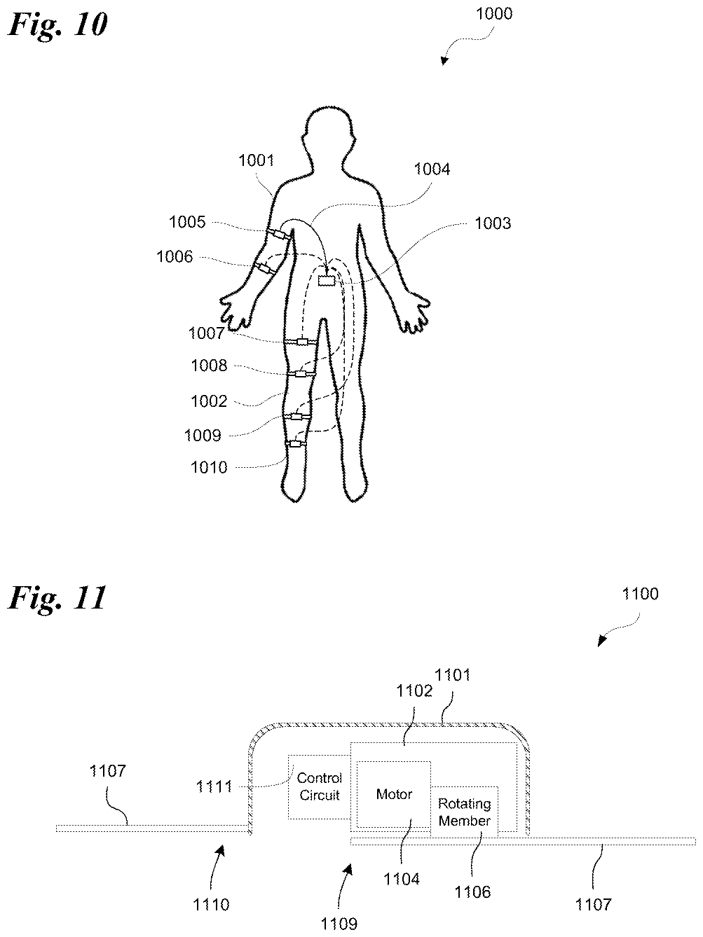

In another aspect illustrated in FIG. 10 at 1000 is an example of the disclosed automatic retention system is shown where multiple automatic retention apparatuses of the present disclosure 1005-1010 are mounted adjacent one or more limbs such as an arm 1001, or a leg 1002. In this example, at least one automatic retention apparatus (e.g. 1005) of the multiple automatic retention apparatuses 1005-1010 is mounted remote from a control circuit 1003. In this example, control circuit 1003 is optionally connected to one or more automatic retention apparatuses. The individual automatic retention apparatuses may thus optionally operate without individual control circuits but may rely instead on a centrally located control circuit or circuits to coordinate the actions of some or all of the individual retention apparatuses 105-1010.

The individual retention apparatuses in the centralized control circuit may communicate using one or more communication links like communication link 1004. In one example, control circuit 1003 is optionally a single control circuit operatively coupled to multiple automatic retention apparatuses 1005-1010 using multiple communication links. These communication links may be wired thus using one or more cables to carry electrical signals from control circuit 1003 to the one or more automatic retention apparatuses 1005-1010. In another example, one or more of the communication links may be wireless optionally employing a transmitter and receiver in the communication circuitry for the automatic retention apparatuses 1005-1010 and in the control circuit 1003. These communication links may optionally be configured to carry electrical signals defining commands indicating which of the multiple apparatuses should apply pressure, and how much. The automatic retention apparatuses may optionally send return signals defining the state of the individual apparatuses, results of specific commands, or other aspects related to the operation of the individual apparatuses 1005-1010.

In another aspect, the individual automatic retention apparatuses of the present disclosure may optionally include, or be mounted to, an individual housing, and at least one control circuit may be mounted inside the housing. As illustrated at 1100 in FIG. 11, a control circuit 1111 is optionally mounted within an individual housing 1101. An actuator 1102 may be optionally mounted in a housing 1102, and the actuator may include a motor 1104 coupled to a rotating member such as a rotating member 1106. The rotating member 1106 may be coupled to motor 1104 by any suitable arrangement of devices for transferring torque from the motor 1104 to the rotating member 1106 such as by shafts, linkages, belts, chains, gears, and the like. In this example, the rotating member 1106 extends out of the housing to engage the retention member 1107 at an engagement region 1108 that is at, or adjacent to, a first end 1109 of the retention member. A second end 1110 is optionally mounted to the housing. In another aspect, housing 1101 and the second end 1110 may optionally be fused together, or formed as a single unitary structure.

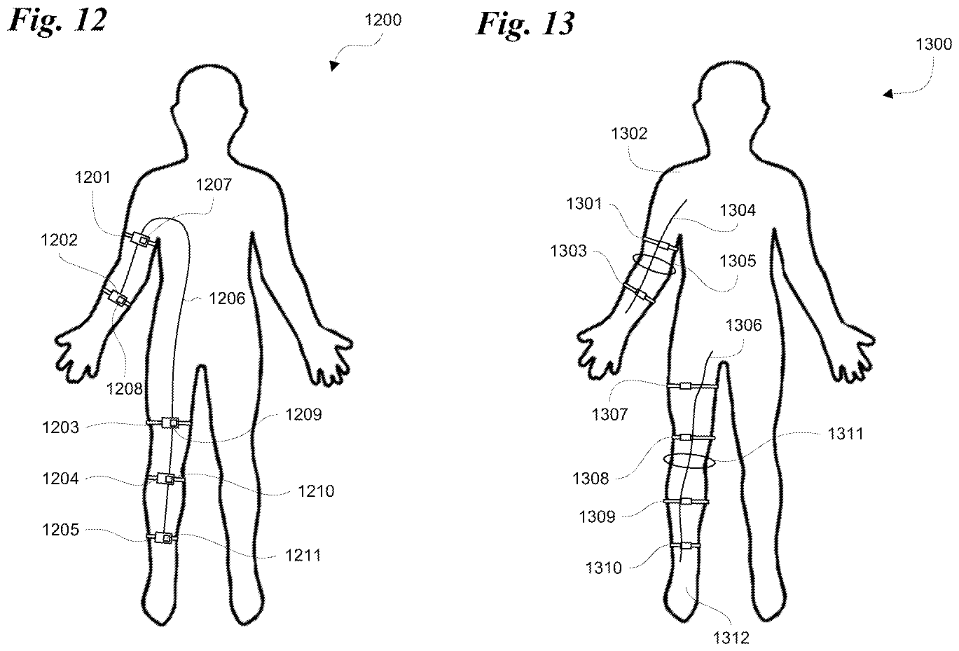

In this example, one or more, or all, individual automatic retention apparatuses of the present disclosure may individually include a separate control circuit which may be configured to drive an actuator 1122 engage retention member 1107. In another aspect illustrated at 1200 in FIG. 12, the system optionally operates according to processing provided by multiple individual control circuits. The multiple automatic retention apparatuses 1201-1205 may each have a control circuit 1207-1211. These individual retention apparatuses 1201-1205 are optionally separately responsive to one or more separate control circuits 1207-1211. In this example, multiple automatic retention apparatuses with individual control circuits may communicate with one another as peers.

The control circuits may be in communication with and optionally responsive to at least one other control circuit of the multiple individual control circuits. In this configuration, no centralized control circuit is required as all of the individual control circuits may be configured to work together to cooperatively determine which of the control circuits should activate the separate individual automatic retention apparatuses, and to what extent. In another aspect, the multiple individual retention apparatuses may communicate with one another via a communication link 1206. This communication link is illustrated in the abstract at 1206 signifying that all of the retention apparatuses are able to communicate with each other and may or may not be interpreted as a communication link with only a single cable running between the retention apparatuses. This link may be implemented using a cable or wire, or as a wireless communication link between cooperating control circuits. In another aspect, the communication link 1206 may be configured to communicate information between the apparatuses over a shared electrical connection such as via a shared data bus where some or all signals sent between any of the communication circuits may be accessible by all other communication circuits.

In another aspect, communication link 1206 may be implemented by each individual communication circuit communicating with other circuits wirelessly. In this configuration, each automatic retention apparatus may be responsive to every other retention apparatus using a wired or wireless connection operating like a “peer to peer” network configuration. Any suitable communication arrangement such as a bus, ring, or tree topology, using any suitable protocol may be implemented by a wired or wireless configuration (including optical fibers, or other data transmission means) by which the control circuits may be able to communicate with one another.

In another aspect illustrated in FIG. 13 at 1300, multiple automatic retention apparatuses of the present disclosure may include an upper retention apparatus 1301 which may be mounted at an upstream location of a limb 1302. A separate lower retention apparatus 1303 is optionally mounted at a downstream location of the limb. In this example, “upstream” and “downstream” generally refers to the flow of blood traveling through a blood vessel 1304 and may also refer to the flow of blood either away from the heart in the case of an artery, or toward the heart in the case of a vein. Thus downstream in an artery refers to blood flowing away from the heart, and downstream in the case of a vein refers to blood flowing towards the heart. So for example, if blood vessel 1304 is an artery, then retention apparatus 1301 may be thought of as being upstream of retention apparatus 1303. If blood vessel 1304 is a vein, then retention apparatus 1301 may be thought of as being downstream of retention apparatus 1303. In this example, the upstream and downstream portions of the limb 1302 are coupled together by a joint 1305, which is here an elbow joint.

In another aspect, the control circuit or circuits of the present disclosure that are actively controlling the operation of automatic retention apparatuses 1301 and 1303 may be configured with location information such as whether the individual retention apparatuses are upstream or downstream of each other. This configuration information may be useful for the control circuit or circuits in coordinating a lifesaving response to a an injury that occurs in the limb 1302.

For example, if 1304 is an artery, and a sensor in retention apparatus 1301 detects adequate blood pressure in 1304, but a similar sensor in retention apparatus 1303 detects a significant reduction in blood pressure in 1304, the control circuit(s) controlling the operation of these two automatic retention apparatuses may deduce that a significant injury to 1304 has occurred between 1301 and 1303 because of the relative position of each apparatus with relation to the blood flow through the vessel. The system may respond by directing apparatus 1301 to apply significant pressure to blood vessel 1304 by increasing the compression forces applied by the retention member in apparatus 1301. The system may also determine that little if any compression forces should be applied by 1303 given that 1303 is downstream from retention apparatus 1301 with respect to an artery.

Similarly, the multiple automatic retention apparatuses 1307-1310 may be mounted to a limb 1312, which in this example is a leg. Retention apparatuses 1307 and 1308 are here mounted to an upper portion of the leg 1312 and are downstream from automatic retention apparatuses 1309 and 1310 relative to a blood vessel 1306, a vein. Retention apparatuses 1309 and 1310 are here mounted to a lower portion of leg 1312. The upper and lower portions of the leg 1312 are coupled together by a joint 1311, a knee this example. As with the example given above, the automatic retention apparatuses 1307-1310 may operate in concert to adjust compression forces on blood vessel 1306, and may collaborate to deduce the proper response based on their position relative to joint 1311, blood vessel 1306, and to one another.

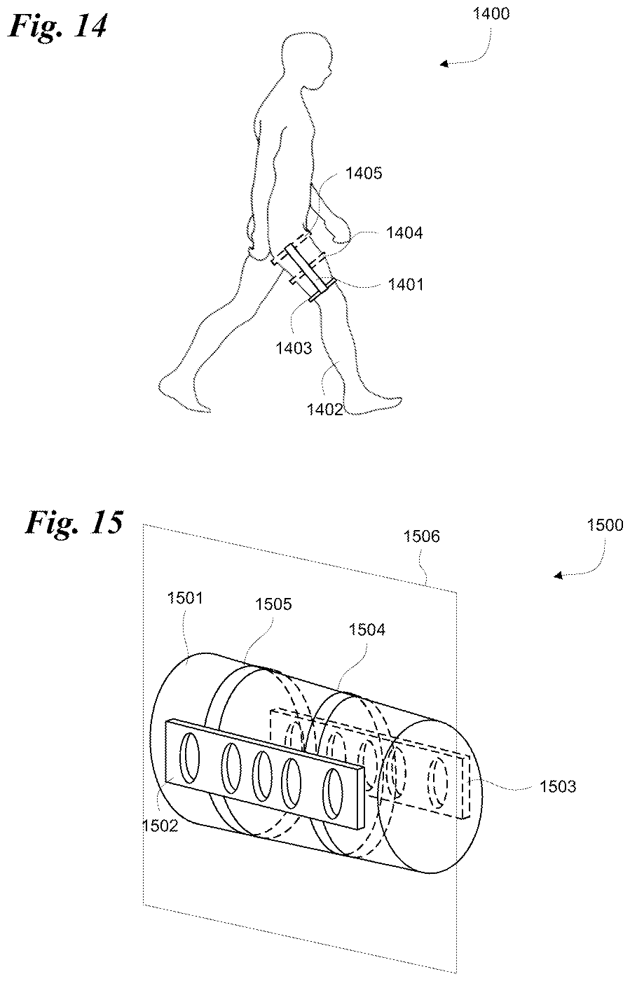

In another aspect illustrated in FIGS. 14-17, the automatic retention system of the present disclosure optionally includes frames, braces, splints, or other such rigid, or semi rigid support members. The automatic retention apparatuses of the present disclosure may be mounted to, or formed as a part of these various types of supporting structures thus providing a combination of support along with coordinated automatic control of compression forces.

In one aspect illustrated at 1400 in FIG. 14, the disclosed automatic retention system may include a frame 1401 configured to receive at least a portion of a limb 1402, in this case a leg. In another aspect, at least one of the multiple retention members 1403 may be mounted to the frame 1401. Other retention members 1404, and 1405 may be mounted to the frame as well. In general, a frame like 1401 may include any suitable number of retention members. Retention members 1403-1405 optionally are controlled by one or more control circuits and actuators as discussed herein elsewhere.

In another aspect, a frame may include multiple frame members. As illustrated at 1500 in FIG. 15, the disclosed automatic retention system may include two frame members 1502, and 1503, and these may be mounted on opposing sides of a limb 1501. In another aspect, the frame members 1502, and 1503 are optionally longitudinally aligned with a reference plane 1506.

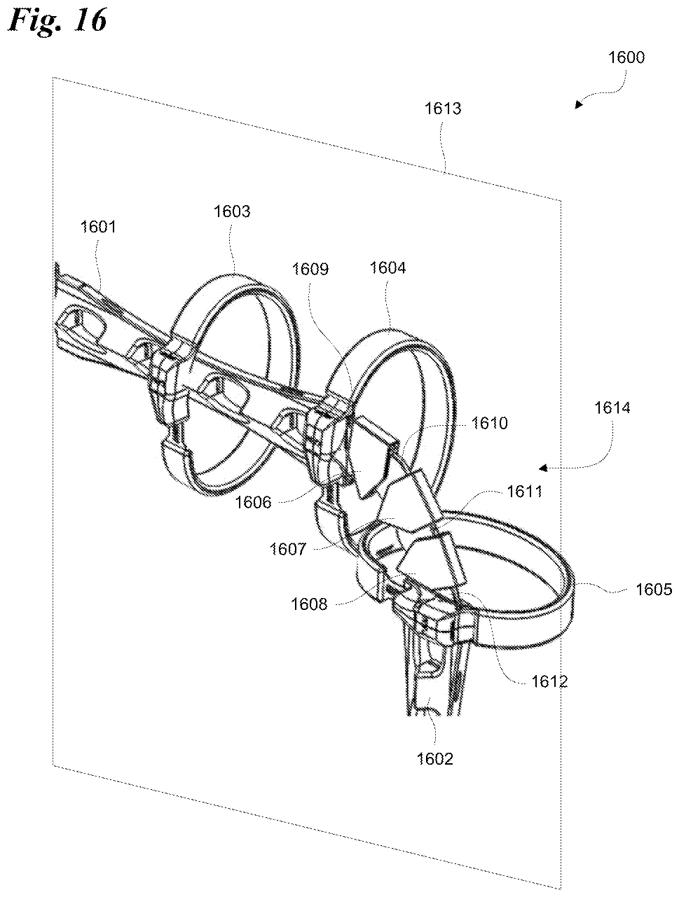

In another configuration illustrated in FIG. 16, the automatic retention system of the present disclosure may include a frame 1600 that optionally includes multiple frame members 1601, and 1602. One or more automatic retention apparatuses 1603-1605 of the present disclosure may be coupled to the frame members 1601 and 1602. In another aspect, the frame 1600 may be integrated into a garment such as the garment(s) discussed herein elsewhere. For example, frame 1600 may be mounted outside a garment, may be incorporated into the garment, or mounted to a limb and the garment placed over frame 1600.

Frame 1600 as illustrated, is optionally configured to resist rotation except along a reference plane 1613. In this way, the frame 1600 may resisting twisting forces applied to a joint of a limb while allowing the limb to function substantially normally. Frame 1600 may include multiple support elements 1606, 1607, and 1608 which may be coupled together, and coupled to the frame members 1601, and 1602. Projecting members 1609-1612, may be interspersed between the support elements 1606-1608, thus optionally creating a joint at 1614 that may correspond to the joint of a limb.

In another aspect, the support elements 1606-1608 may define interior cavities configured to receive the projecting members 1609-1612. In another aspect, the projecting members may be independently rotatable within the cavities defined by the support elements. In another aspect, frame member 1601, and frame member 1602 may be coupled together by the projecting members and support elements and rotatable substantially parallel to a reference plane 1613. The frame members, supporting elements, and projecting members may thus be rotatable along reference plane 1613, and may be prevented from rotating away from the reference plane 1613 by virtue of the multiple projecting members being positioned within the corresponding cavities of the support elements.

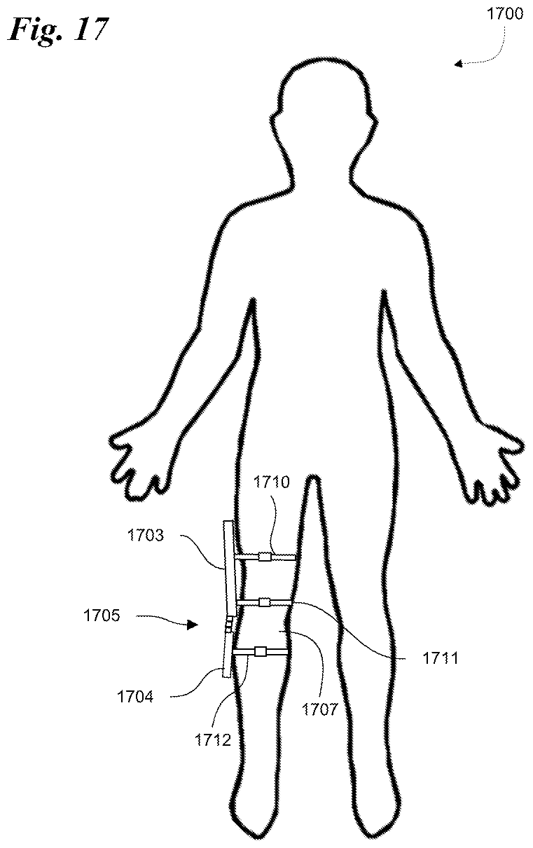

In another aspect, the automatic retention apparatuses 1603-1605 may operate according to the present disclosure to adjust tension on a limb so as to maintain the limb adjacent to frame 1600. In another aspect, the automatic retention apparatuses of frame 1600 may be useful for maintaining proper orientation of the limb with respect to the frame members. For example, as illustrated in FIG. 17, a frame 1705 may include an upper portion 1703 which may be mounted to an upper part of a limb 1708. A lower portion 1704 may be mounted to a lower part of the limb 1708. In this example, the upper and lower parts of the limb 1708 may be coupled together by a joint 1707. At least one upper retention member 1710 is optionally mounted to the upper portion 1703, and surrounds at least a portion of the upper part of the limb. At least one other retention member 1712 is optionally mounted to the lower portion 1704 of the frame, and may be configured to surround at least a portion of the lower part of the limb 1708.

In one example, FIG. 17 illustrates how the frame of FIG. 16 might be applied to a knee joint. The frame 1705 corresponds to frame 1600, and the retention member 1710 corresponds to the retention member of the apparatus 1603, retention member 1711 corresponds to the retention member of apparatus 1604, and retention member 1712 corresponds to the retention member of apparatus 1605. The illustrations provided in FIGS. 17 and 16 are illustrative and not restrictive as the principles illustrated may be applied to any suitable frame and limb arrangement.

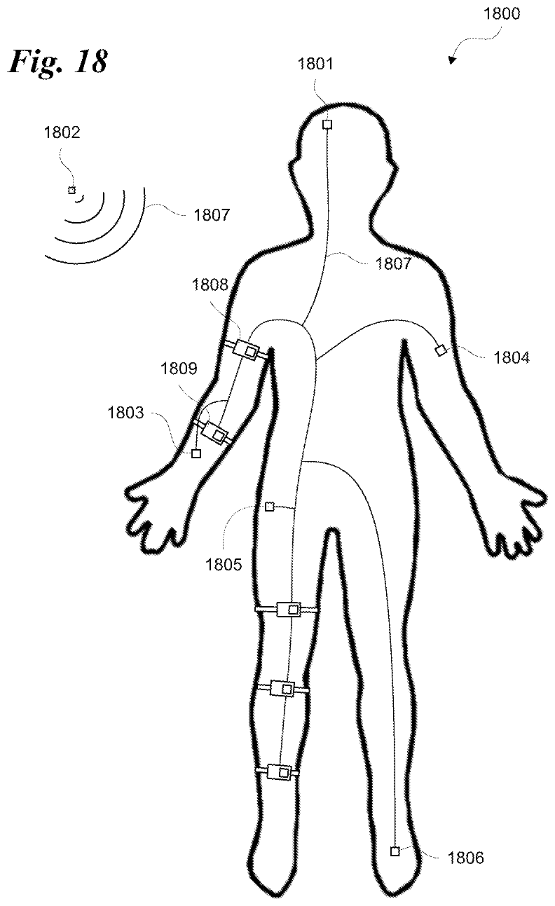

In another aspect illustrated in FIG. 18 at 1800, the sensors of the present disclosure that may be included in the overall system optionally include multiple individual sensors, and the multiple individual control circuits may be separately responsive to these sensors. For example, sensors 1801-1806 may be located in different parts around the body and coupled to one or more automatic retention apparatuses 1808, and 1809. The disclosed sensors may communicate sensor data to the automatic retention apparatuses via a communication link 1807 which may include one or more wired, or wireless communication links. In this way, sensors of the automatic retention system of the present disclosure may be worn by the user, or may be provided in the area of the user, and may automatically communicate with the system to provide sensor input.

Any suitable sensors may be used in the system of the present disclosure. For example, these sensors may include a blood pressure sensor, a temperature sensor configured to determine a temperature of a limb, a heart rate sensor, a temperature sensor configured to determine the ambient temperature around the limb, an accelerometer, ambient air pressure sensor, sensors for measuring electrical activity within the heart, brain, or other area of the body, or any combination thereof.

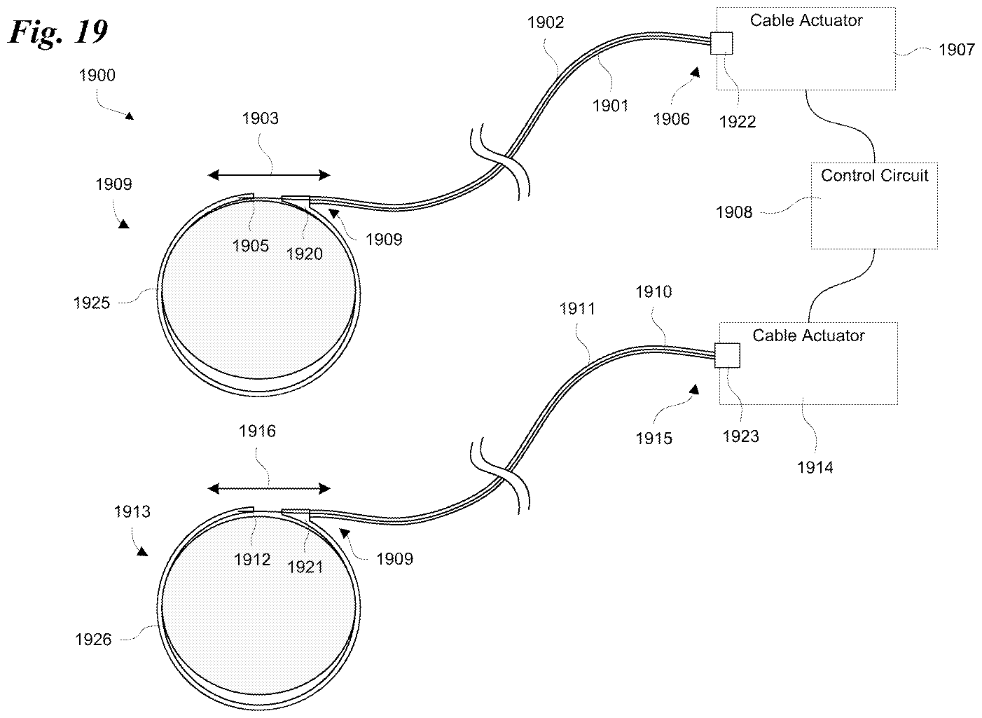

In another aspect, the automatic retention system may include cable actuation whereby an actuator may receive force via a rigid cable and conduit to actuate an automatic retention apparatus, or multiple automatic retention apparatuses. As illustrated in FIG. 19 at 1900, the system may include a cable 1901 inside a conduit 1902. The cable 1901 may be coupled adjacent a first end 1904 to an actuator 1905 of the automatic retention apparatus 1909. A cable actuator 1907 may be responsive to a control circuit 1908 that is coupled to the cable 1901 adjacent a second end 1906 of the cable. The cable 1901 may be selectively movable within the conduit 1902 according to movement initiated by the cable actuator 1901. The actuator 1905 of automatic retention apparatus 1909 is optionally configured to adjust tension on the retention member 1924 according to movement 1903 of the cable 1901 relative to the conduit 1902.

In another aspect illustrated in FIG. 19, multiple cables 1901, and 1910 may be separately positioned inside separate individual conduits 1902, 1911, the multiple cables extending between separate actuators 1905, 1912 of the two different automatic retention apparatuses 1909, and 1913 and the control circuit 1908. Actuator 1912 may be coupled to the cable 1910 adjacent a first end 1917 of cable 1910. A cable actuator 1914 may be responsive to a control circuit 1908 that is coupled to the cable 1910 adjacent a second end 1915 of the cable. The cable 1910 may be selectively movable within the conduit 1911 optionally according to movement initiated by the cable actuator 1914. In this example, multiple cable actuators responsive to the control circuit 1908 may each individually be coupled at a first end 1904, 1916 of each of the multiple cables 1901, 1910. The multiple cables may be selectively movable within the individual conduits according to movement of the cable actuators, and the separate actuators may be coupled to a second end of each of the multiple cables. The separate actuators are optionally configured to adjust tension applied to corresponding retention members 1925 and 1926 according to movement 1903 and 1916 of the individual cables relative to the individual conduits.

In another aspect, the conduit or conduits may be anchored at a first end 1904 and/or 1916 to a first cable mount 1920 and/or 1921, optionally, the conduit is anchored at a second end to a second cable mount 1922 and/or 1923 of the cable actuator(s).

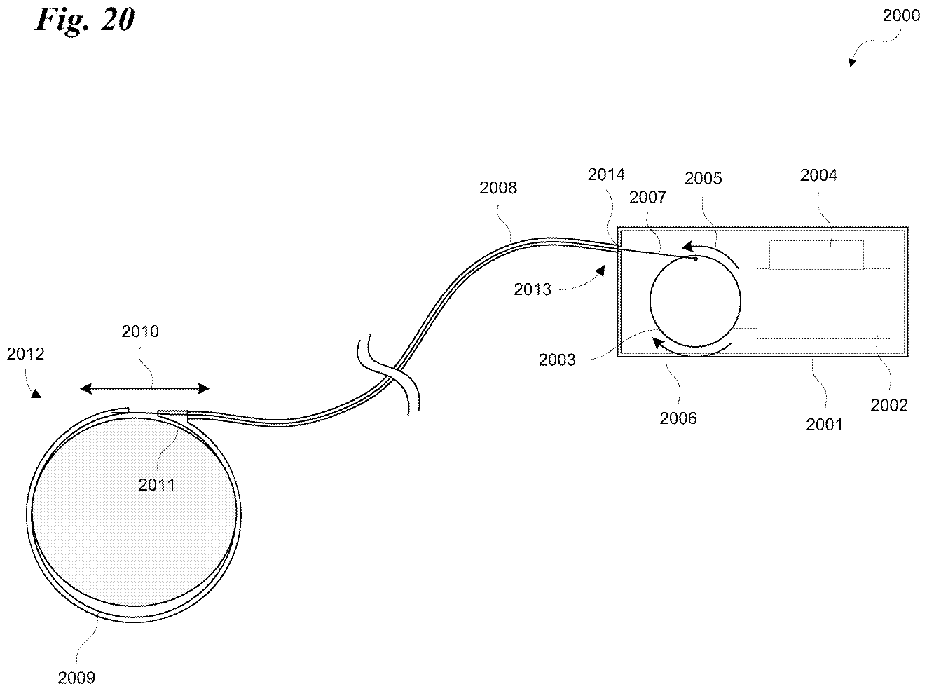

In another aspect illustrated at 2000 in FIG. 20, a cable actuator 2001 optionally includes an electric motor 2002 mechanically coupled to a rotating member 2003. The electric motor may be responsive to control input from a control circuit 2004. The control circuit is optionally programmed to control the electric motor 2002 to rotate in a first direction 2005 and a second direction 2006 opposite the first direction to adjust the position of a cable 2007 relative to the conduit 2008. In this example, rotation in the first direction 2005 results in a reduction in tension on retention member 2009, while rotation of the second direction 2006 results in an increase in tension retention member 2009. In another aspect, the conduit 2008 is optionally anchored at a first end 2010 to a first cable mount 2011 of the automatic retention apparatus 2012. The conduit 2008 is also optionally anchored at a second end 2013 to a second cable mount 2014 of the cable actuator 2001.

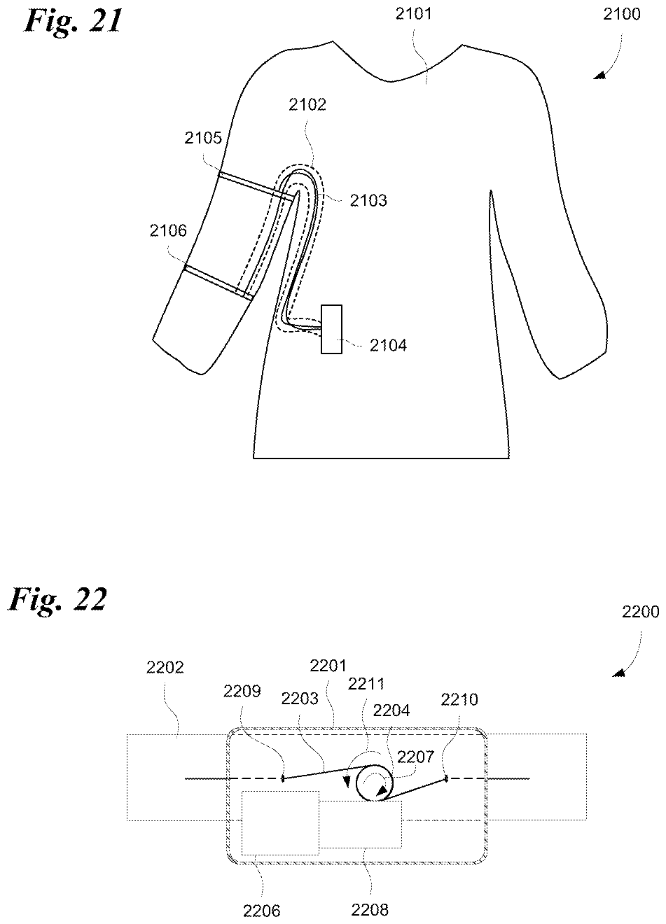

Illustrated at 2100 and FIG. 21, is an example of a garment 2101 of the present disclosure that is optionally configured to surround at least a portion of one of the one or more limbs (in this case arms). Garment 2101 optionally defines an interior passageway 2102 and the cable and conduit 2103 are positioned inside the interior passageway 2102. In this example, a control circuit 2104 with one or more cable actuators of the present disclosure may be positioned within garment 2101 remote from multiple automatic retention apparatuses 2105 and 2106. This example illustrates two separate retention apparatuses with corresponding individual cables, however, any suitable number of interior passageways, cables, and controllers may be used.

In another aspect, the automatic retention apparatuses of the present disclosure may include a winding mechanism for engaging a retention member. An example of this concept is illustrated in FIG. 22 where an automatic retention apparatus 2200 is illustrated having a housing 2201 and an engagement portion 2203 of a retention member 2202. The engagement portion 2203 optionally winds and unwinds around a rotating member 2204 inside the housing. The rotating member 2204 may include a shaft mechanically engaging with an optional gear mechanism 2208 driven by a motor 2206. Tension may be applied to the retention member 2202 when the rotating member 2204 is rotated in a direction 2207 thus winding the engagement portion 2203 of the retention member onto rotating member 2204. Engagement portion 2203 may be a single piece of cable, wire, or any other suitable material, or may comprise multiple pieces such as a separate pieces coupled to rotating member 2204 and extending outwardly to engage retention member 2202. In another aspect, engagement portion 2203 may enter the housing 2201 at an opening 2209 and/or 2210 which may be in a side of the housing 2201 facing the retention member 2202, or on in any other suitable location.

In another aspect, the gear mechanism 2208 may include a worm gear with teeth engaging teeth of the rotating member 2204. Such a configuration may advantageously provide a braking mechanism to reduce or eliminate the opportunity for the rotating member to spin backwards and unintentionally release tension on the engagement portion 2203. In another aspect, the retention member 2202, and/or the engagement portion 2203 may include elastic elements such as elastic bands, springs, rubber bands, or other similar biasing elements to automatically unwind engagement portion 2203 from rotating member 2204 when the rotating member is actuated to reduce tension.

In this example, the actuator of an automatic retention apparatus of the present disclosure includes a rotating member 2204 engaging with at least a portion of the retention member 2202. The rotating member 2204 is optionally rotatable in a first direction 2211 to reduce tension of the retention member 2202, and is optionally rotatable in a second direction 2207 opposite the first direction to increase tension of the retention member 2202. The actuator may include motor 2206, and/or gear mechanism 2208 and may be mechanically coupled to the rotating member 2204. The actuator may be arranged and configured to rotate the rotating member 2204 in the first direction and the second directions to increase or decrease tension of the retention member in response to input from a control circuit of the present disclosure. In another aspect, the portion of the retention member 2203 that engages the rotating member 2204 is optionally arranged and configured to wrap around the rotating member 2204 as it rotates.

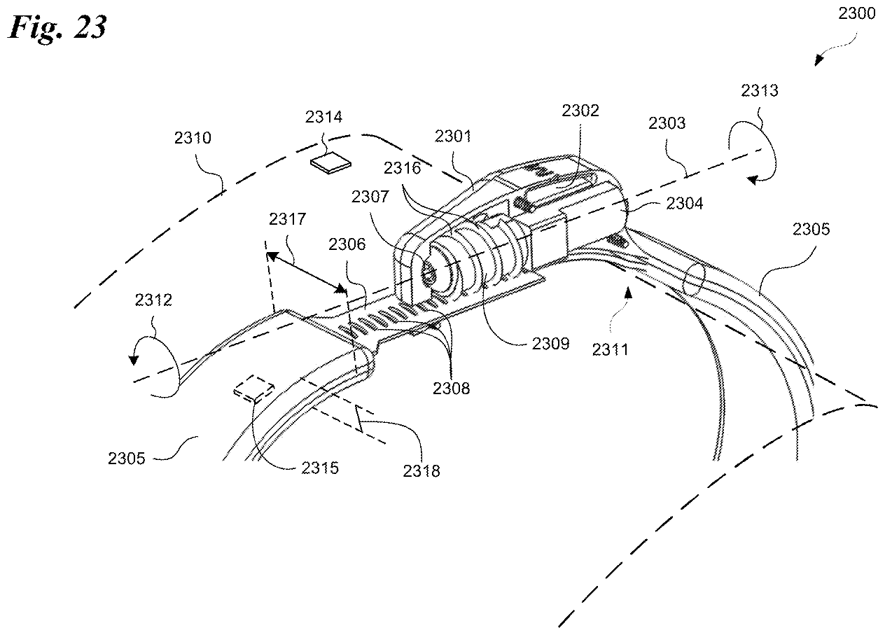

FIG. 23 illustrates other aspects which may be incorporated in the disclosed examples of an automatic retention apparatus. An exemplary automatic retention apparatus 2300 is shown for automatically adjusting pressure on an object 2310. A retention member 2305 may be included that is optionally separate and distinct from object 2310, and may be positioned around the object. The retention member 2305 may be arranged and configured to engage the object 2310 to increase or decrease pressure applied to the object according to the present disclosure.

The retention apparatus 2300 may include a housing 2301 separate and distinct from the object 2310, and the retention member 2305 may be mounted to the housing by any suitable means, one example of which is shown at 2311 where the retention member is bonded to the housing. Any suitable bonding technique may be employed such as by fasteners, adhesives, solvents, ultrasonic welding, or chemical bonding to name a few nonlimiting examples. In another aspect, the mounting at 2311 may be achieved by forming the housing 2301 and the retention member 2305, or a portion thereof, as a single unitary structure.

An actuator may be mounted inside the housing 2301 that optionally includes a motor 2304 coupled to a rotating member 2309 that optionally rotates on a shaft 2307. The rotating member 2309 may be positioned to engage the retention member 2305. In this example, the rotating member includes a worm gear that optionally extends out of the housing 2301 toward the object 2310 to engage an engagement portion 2306 of the retention member 2305 adjacent to the housing. Engagement portion 2306 includes one or more grooves, recesses, or openings 2308 defined by the engagement portion 2306. Grooves 2308 engage one or more teeth 2316 of the rotating member 2309. The rotating member 2309 is optionally rotatable around an axis of rotation 2303 that may be substantially parallel to the retention member 2305 wrapped around the object 2310.

In another aspect, using a worm gear for the rotating member 2309, or for other examples of a rotating member of the present disclosure, where the teeth of the gear engage grooves like grooves 2308, may advantageously provide a braking mechanism without additional wear or power usage. Using a worm gear may reduce or eliminate the opportunity for the rotating member to spin backwards thus unintentionally releasing tension on the engagement portion 2306 and the retention member 2305 in general.

The rotating member 2309 may be rotated by a motor 2304 controlled by a control circuit 2302 of the present disclosure. The rotating member 2309 may rotate in a first direction 2312 to increase tension on the retention member 2305, and in a second different direction 2313 to decrease tension on the retention member.

The automatic retention apparatus may include at least one sensor 2314 and/or 2315 of the present disclosure which may be arranged and configured to sense changes in a sense parameter associated with the object 2310. Control circuit 2302 may be included that is optionally responsive to input from the sensor(s) 2314 and 2315. The control circuit 2302 may be configured to control the actuator according to the input from the sensors as disclosed herein, the control circuit being configured to control the motor 2304 to actuate the rotating member 2309 to rotate it in the first or second direction 2312 and 2313 respectively to adjust tension on the retention member based on input from the sensor

The sense parameters sensed by sensors 2314 and 2315 may be any parameters of interest in determining when, and to what extent, the tension on retention member 2305 should be adjusted. In the case where the object 2310 is a human or animal appendage, example sense parameters include, but are not limited to, body temperature, heart rate, nearby blood flow rate, nearby blood pressure, blood oxygen, perspiration, respiration rate, or electrical or chemical impulses related to heart beat, stress, emotion, pain, and the like. In another example, sensor 2315 may be located on, or adjacent to grooves 2308, or positioned as a linkage between portions of retention member 2305 where the sensor may operate as part of the retention member.

In another aspect, sensor 2314 may be mounted to object 2310 separate from the housing 2301, and may be configured to establish and maintain a communication link between sensor 2314 and control circuit 2302. In another aspect, sensor 2315 may be mounted to, or included as part of, retention member 2305 and may obtain sensor input from object 2310 by virtue of close proximity of the retention member 2305 to the object 2310.

The sensor 2315 may be thought of as a first sensor mounted to a retention member 2305 and may be configured to generate input based on tension in the retention member. Sensor 2314 may be thought of as a second sensor mounted to an object 2310 the retention member at least partially surrounds. The one sensors may be configured to generate input based on movement of the object 2310.

In another aspect, the retention member 2305 illustrated in FIG. 23 is optionally substantially rigid and relatively inflexible. The retention member 2305 may also define a width 2317 and a thickness 2318, and in some examples, the retention member may be wider than it is thick. That is to say, dimension 2317 may be greater than dimension 2318 allowing for retention member 2305 to optionally be thin and flat relative to its length. The retention member optionally an elongate retention member, and the projections and recesses may be defined in a portion of the retention member that is wider than it is thick, and narrower than it is long.

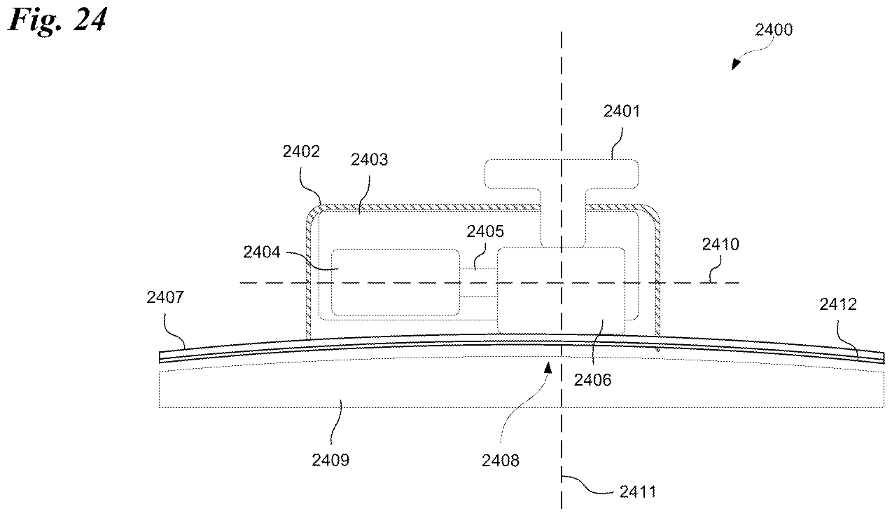

FIG. 24 illustrates an actuator of the present disclosure that includes a manual control mechanically coupled to a rotating member of an actuator. In this example, the manual control is optionally arranged and configured to rotate a rotating member in a first and second direction to adjust tension of a retention member based on input from a user. An automatic retention apparatus 2400 optionally includes a housing 2402. In this example, the retention member 2407 optionally passes through the housing 2402, or optionally passes into an out of the housing through an opening defined by the housing. Any configuration of engagement between the retention member of the present disclosure and an actuator is envisioned (e.g worm gear, windlass, capstan, electric motor, etc.)

In another aspect, a protective layer 2412 is optionally positioned between the retention member 2407 and the object 2409 that pressure is applied to by retention apparatus 2400. This protective layer 2412 may be implemented as a protective sleeve the retention member 2407 passes through, as a layer of material applied retention member 2407, as a portion of a garment the retention member 2407 may be passing through, or any combination thereof. In one aspect, the protective layer 2412 has a coefficient of friction that is less than the coefficient of friction for the retention member 2407. In another aspect, the retention member 2407 may move laterally across the protective layer 2412 as the tension is applied to the retention member. In another aspect, the protective layer 2412 may experience little or no movement with respect to the object 2409 while tension is applied to retention member 2407.

An actuator 2403 is optionally mounted inside the housing 2402, and optionally includes a rotating member 2406 that may be positioned to engage the retention member 2407 at an engagement region 2408. In another aspect, the rotating member 2406 optionally engages the retention member 2407 inside the housing 2402, such as in the case of where the retention member 2407 passes into the housing through an opening. The retention member 2407 may pass through one or more holes defined by the housing 2402 to enter the housing from the sides, or in another example, in one or more holes defined on the bottom of the housing.

A motor 2404 may be included and coupled to the rotating member by a connecting member 2405 (e.g. a shaft, linkage, belt, chain or other suitable connecting member). In one example, the rotating member 2406 may be rotatable around an axis of rotation 2410 in a first direction to increase tension on the retention member 2407, and in a second direction to decrease tension on the retention member. In another example, the rotating member 2406 is optionally rotatable around an axis of rotation 2411 in a first direction to increase tension on the retention member 2407, and in a second direction to decrease tension on the retention member.

A manual control 2401 is optionally included in the automatic tensioning apparatuses of the present disclosure for manually adjusting tension on the retention member 2407 based on input from a user or operator. In this example, manual control 2401 is operable as a user interface for taking input from a user adjusting the operational characteristics of the automatic tensioning apparatus 2400. Rotating the manual control 2401 results in rotation of the rotating member 2406, thus allowing for an alternative means of adjusting the rotating member 2406 where a motor 2404 is absent, or where the motor 2404 is malfunctioning. In another aspect, manual control 2401 may provide the exclusive means for providing torque on retention member 2407. In this example, torque may be applied by rotating the manual control where the electric motor 2404 and the linkage 2405 are absent.

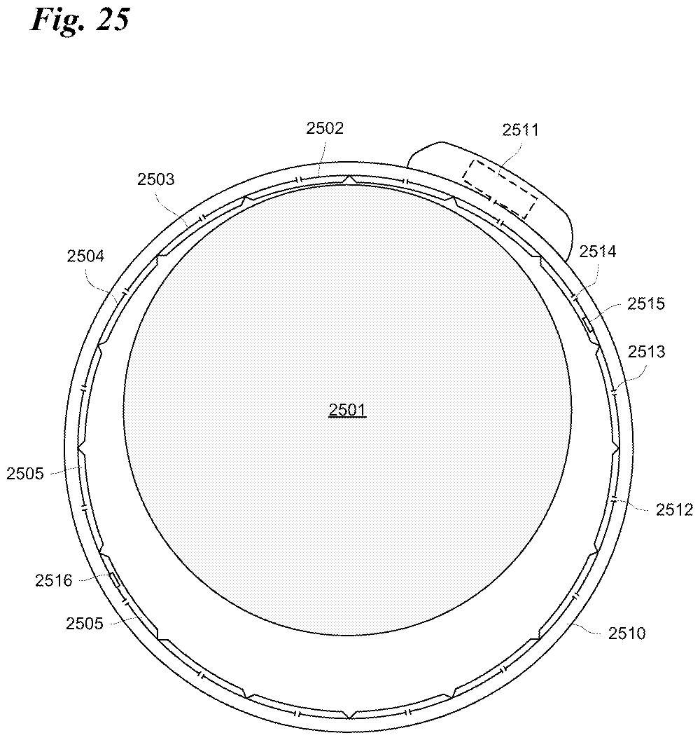

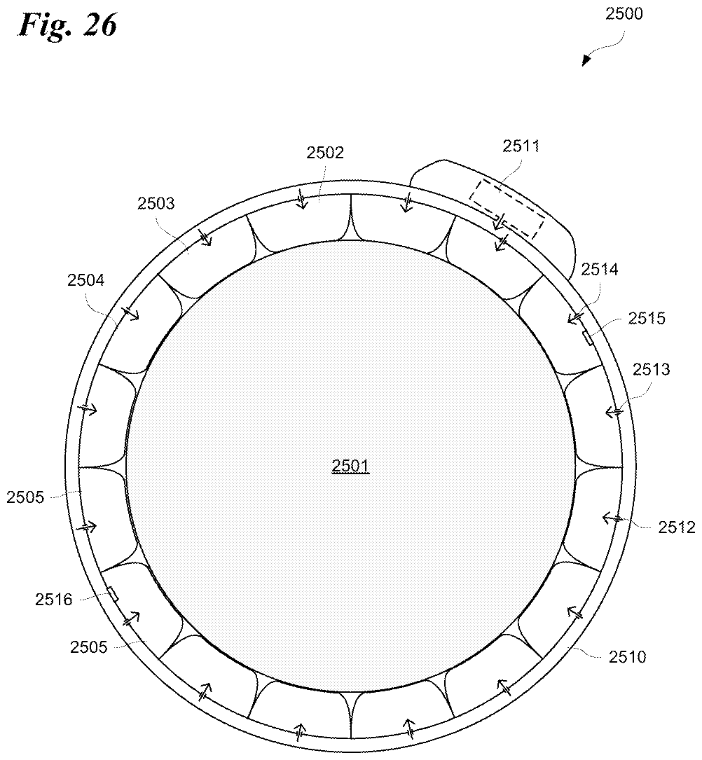

Another example of the disclosed automatic retention apparatuses is illustrated in FIGS. 25 and 26. In this example, the automatic retention apparatus 2500 applies pressure to an object 2501 according to the inflation state of one or more inflatable cavities 2502-2506 positioned between a retention member 2510 and the object. In this example, an actuator is optionally arranged and configured to increase or decrease a fluid pressure within the individual cavities to inflate them to adjust the compression forces applied to the object by the automatic retention apparatus. In another aspect, the inflatable cavities are optionally in fluid communication with each other, and may thus inflate or delate together. In another aspect, groups of cavities may be in fluid communication with one another, but separate groups of cavities may not be in fluid communication with other groups.

Automatic retention apparatus 2500 optionally includes retention member 2510 surrounding a portion of object 2501, which may be any object, such as a portion of a limb or appendage of a human or animal subject. One or more inflatable cavities 2502-2506 may be positioned between the retention member 2510 and the object 2501. In FIG. 25, the cavities 2502-2506 are illustrated in the partially or completely deflated state. In FIG. 26, automatic retention apparatus 2500 is illustrated with 2502-2506 in the partially or completely inflated state during or after the introduction of a fluid into the individual cavities thus increasing their internal pressure relative to the environment outside the cavities.

An actuator 2511 is optionally arranged and configured to inflate the one or more inflatable cavities to adjust pressure applied to the object 2501. Actuator 2511 may be operable to introduce any fluid into the cavities such as water, oil, expanding foam, air, carbon dioxide, nitrogen, argon, or any other suitable gas, and any mixture thereof. In another aspect, sensors of the present disclosure such as those discussed with respect to FIG. 1 and elsewhere herein may be included or accessed by the apparatus 2500 and may be arranged and configured to sense changes in a sense parameter associated with the one or more limbs. The automatic retention apparatus 2500 may include, or be responsive to, a control circuit responsive to this sensor input, and is optionally configured to control the actuator 2511 to adjust inflation of the inflatable cavities according to input received from the sensors. In another aspect, actuator 2511 may include a compression apparatus configured to introduce fluids into the cavities.

In another aspect, the inflatable cavities 2502-2506 are optionally separate and distinct, and the actuator 2511 is optionally configured to selectively inflate a first cavity 2502 at a first pressure, and a second cavity 2506 at a second pressure that is different than the first pressure. For example, separate conduits may be provided to each of the individual cavities, or to separate groups of multiple cavities so that individual inflation pressure of each cavity, or each group of cavities, may be specifically managed by the actuator. In another aspect, fluid entering the cavities may pass through ports such as ports 2512, 2513, and 2514. These ports may be configured individually, or in groups, with pressure limiting devices so that the cavity, or groups of cavities, may accept or retain different levels of fluid pressure. Thus individual cavities may automatically receive different pressure levels, and these pressure levels may be specific to a location of the individual cavity, or group of cavities, with respect to the object.

For example ports 2512-2514 may include a one way valve configured to open when the actuator introduces fluid into the cavities, and to otherwise automatically close to retain the fluid in the cavities. In another aspect, some of the cavities may be configured with different fluid ports that may or may not include one-way valves, pressure limiting devices, or other aspects that may change their rate of inflation, or the passage of fluid into an out of the cavity with respect to other cavities. Thus some cavities may be configured to inflate faster, and/or deflate slower with respect to the cavities around them.

As illustrated, the inflatable cavities are optionally arranged circumferentially around the inside of a retention member 2510. However, the inflatable cavities may be arranged in any suitable configuration such as with cavities running longitudinally. In another aspect, sensor input may include a sensor 2515 and/or 2516, or others, mounted to the retention member 2510 of the automatic retention apparatus 2500. These sensors may be configured to generate input based on an interior pressure of at least one of the one or more inflatable cavities. Sensors may be included in every cavity, or may be limited to individual groups of cavities. Such sensor input may be used to augment sensory input received from other sensors disclosed herein elsewhere that are configured to monitor the environment, or aspects of the object 2501.

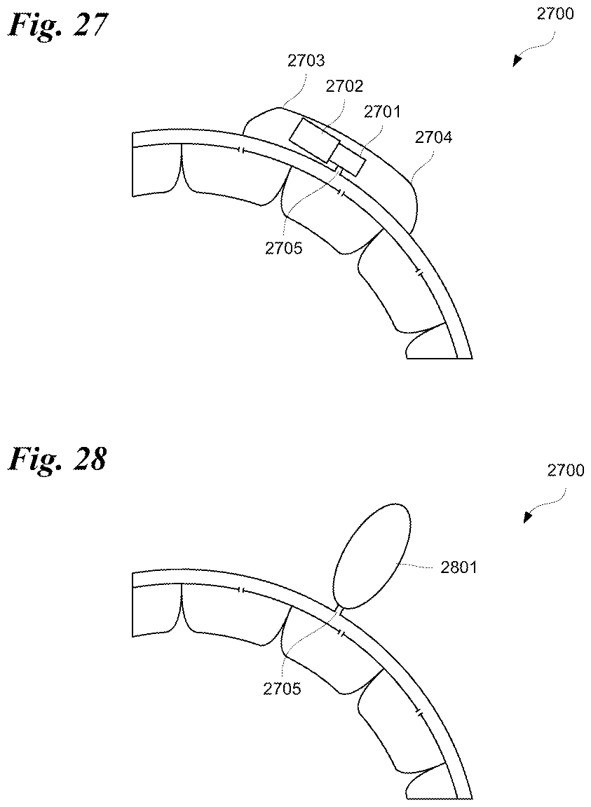

In another aspect illustrated at 2700 in FIG. 27, the automatic retention apparatus includes a fluid compression apparatus that is optionally an air compressor 2701 responsive to a control circuit of an actuator 2702, or of any control circuit disclosed herein. In one aspect, actuator 2702, and compressor 2701 may be mounted in a housing 2703. The housing 2703 may be coupled adjacent to the retention member 2704 So that an output port from the compressor 2701 is aligned with and coupled to an input port 2705 of the retention member.

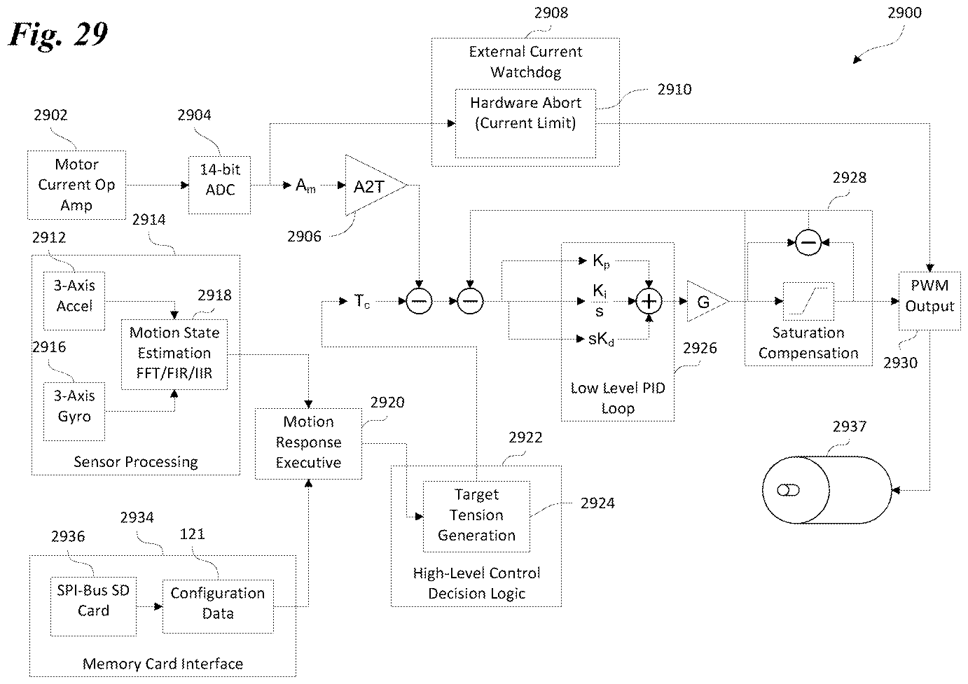

The fluid compressed by compressor 2701 may include air, carbon dioxide, nitrogen, argon, or any other suitable gas, and any mixture thereof. In another aspect, the fluid pressure may be provided by a fluid in the liquid state such as water, oil, or any other suitable liquid. In another aspect illustrated in FIG. 28, the automatic retention apparatus 2700 is optionally configured to receive compressed air from a reservoir 2801 rather than from compressor 2701. In one example, housing 2703 may be removed and reservoir 2801 may be used in its place to provide the internal fluid pressure within the individual cavities as illustrated in FIGS. 25 and 26. In another aspect, the reservoir may include compressed carbon dioxide gas, or a liquid such as oil or gas, or the reservoir may include a squeeze bulb. In another aspect, the reservoir, compressor, or other fluid actuator may be remote from the retention apparatus and coupled to the apparatus via a conduit for carrying the fluid pressure applied to the interior of the cavities.

In operation, the control circuit and/or other electronics in the various examples of an automatic retention apparatus disclosed herein is operable to automatically adjust the tension on the retention member. In one operational aspect, the control circuit is programmed to perform a power on process for the data collection and control electronics. The process may begin by receiving a power on command to activate the apparatus including the control circuit and any additional control electronics. The control circuit may initiate communication with an inertial sensor suite via a digital interface, and may also initialize a file system in the memory for recording data and maintaining configuration data such as the configuration data discussed herein. The control circuit may also begin calibration of all available sensors such as any inertial sensors. This may include configuring the resolution of the sensors and the sample rate. It may also include configuring sensor noise filter.

The control circuit may also be configured to execute a data collection and control algorithm. The algorithm may include retrieving the available stream of data from any available sensors representing the values of the various sense parameters generated by the sensors. The control circuit may apply/update a digital filter of state data, and/or use an adaptive algorithm such a neural network, or similar algorithm to identify important data features in the time and frequency domain of the incoming data stream. The control circuit may use the resulting data, configuration parameters, and real-time data features to calculate one or more values representing the tension to be applied to the retention member. The control circuit may compare the values to measured device parameters and communicate the tension values to the actuator to adjust the tension accordingly. The data collection and control algorithm may then repeat as necessary. The algorithm may execute multiple times a second such as more than 10 times a second, more than 1000 times a second, or more than a million times a second.

One example of circuit components for processing signal input and producing a motor control output is illustrated at 2900 in FIG. 29. These components may be used with, or included in components discussed herein elsewhere, particularly with respect to the components illustrated in FIG. 1 at 100. The control circuit at 2900 may include multiple sub circuits such as a sensor processing circuit 2914, a memory card interface 2934, and high-level control of decision logic circuit 2922, and external current watchdog circuit 2908, a low level Proportional Integral Derivative (PID) loop 2926, and the saturation compensation circuit 2928. External current watchdog circuit 2908 may include a hardware abort aspect which may operate as a current limiter to avoid overloading motor 2937. A motor current operational amplifier (or “Op Amp”) 2902 passes signals representing the data value for motor current to a 14-bit Analog to Digital Converter (ADC) 2904. The sensor processing circuit 2914 may include any suitable sensors like sensors 115 that may include a 3-axis accelerometer 2912 and 3-axis gyro 2916 which may be used to estimate the motion state at 2918 by utilizing such filters as the FFT (Fast Fourier Transform), FIR (Finite Impulse Response), and IIR (Infinite Impulse Response). Memory card interface 2934 may include an SPI-bus and SD card reader 2936 with access to update configuration data 2935 which may include user configurable aspects or operating parameters of the automatic retention apparatus. The motion response executive 2920 then reads the motion state and configuration data and passes the result to high-level control decision logic 2922 which may then determine a target tension using target tension generation circuits 2924. This target tension may be compared to the actual tension calculated from motor current or sensor circuitry comparing aspects such as force, torque or position data. The result is passed to PID loop 2926 and saturation circuitry 2928 producing an output such as Pulse Width Modulation (PWM) output 2930 which may be provided to motor 2937 to automatically control tension on the retention member as discussed herein elsewhere.

Other disclosed concepts include the following numbered examples:

### Example 1

An automatic retention system for applying pressure to one or more limbs of a human or animal, the system including: multiple automatic retention apparatuses that optionally include: a retention member surrounding a portion of at least one limb of the one or more limbs; and an actuator arranged and configured to engage the retention member, wherein the actuator is configured to actuate the retention member to adjust tension applied to the at least one limb by the retention member; at least one sensor arranged and configured to sense changes in a sense parameter associated with the one or more limbs; and at least one control circuit responsive to the at least one sensor, wherein the at least one control circuit is configured to control the actuators of the multiple retention apparatuses to adjust tension applied to the at least one of the one or more limbs according to input received from the at least one sensor.

### Example 2

The automatic retention system of any preceding example, the system also including a garment configured to surround at least a portion of one of the one or more limbs.

### Example 3

The automatic retention system of any preceding example, wherein at least one of the multiple automatic retention apparatuses is positioned outside a garment.

### Example 4

The automatic retention system of any preceding example, wherein at least one of the multiple automatic retention apparatuses is positioned within a cavity defined within a garment.

### Example 5

The automatic retention system of any preceding example, wherein a retention member of at least one of the multiple automatic retention apparatuses is woven into a garment.

### Example 6

The automatic retention system of any preceding example, wherein a garment includes an actuator mount configured to couple an actuator of at least one of the multiple automatic retention apparatuses adjacent to the retention member.

### Example 7

The automatic retention system of any preceding example, wherein a retention member of at least one of the multiple automatic retention apparatuses includes a cloth strap that is mounted to the garment at a predetermined mount location.

### Example 8

The automatic retention system of any preceding example, wherein a cloth strap is mounted inside a garment, wherein the garment defines an opening through which the actuator engages the retention member to adjust tension on the cloth strap.

### Example 9

The automatic retention system of any preceding example, wherein a garment defines an interior passageway and at least a portion of one retention member of the multiple automatic retention apparatuses is positioned inside the interior passageway

### Example 10

The automatic retention system of any preceding example, wherein the actuator of the at least one retention member is mounted outside an interior passageway.

### Example 11

The automatic retention system of any preceding example, wherein a garment includes multiple mounts corresponding to each of the multiple automatic retention apparatuses.

### Example 12

The automatic retention system of any preceding example, wherein at least one automatic retention apparatus of the multiple automatic retention apparatuses is mounted remote from the at least one control circuit.

### Example 13

The automatic retention system of any preceding example, wherein at least one automatic retention apparatus of the multiple automatic retention apparatuses is mounted to a housing and the at least one control circuit is mounted inside the housing.

### Example 14

The automatic retention system of any preceding example, wherein an upper retention apparatus of the at least one automatic retention apparatuses is mounted at an upstream location of a limb of the one or more limbs, and a separate lower retention apparatus of the at least one automatic retention apparatuses is mounted at a downstream location of the limb.

### Example 15

The automatic retention system of any preceding example, wherein upstream and downstream portions of a limb are coupled together by a joint of the limb.

### Example 16

The automatic retention system of any preceding example, the system also including a frame configured to receive at least a portion of a limb of the one or more limbs, wherein at least one of the multiple retention members is mounted to the frame.

### Example 17

The automatic retention system of any preceding example, wherein a frame is included that has two frame members on opposing sides of the limb, and wherein the two frame members are longitudinally aligned with a reference plane.

### Example 18

The automatic retention system of any preceding example, the system also including multiple support elements coupled together and coupled to a frame, the multiple support elements also aligned with a reference plane, wherein the multiple support elements and the frame are coupled together and rotatable substantially parallel to the reference plane, and are prevented from rotating away from the reference plane by virtue of multiple projecting members positioned within corresponding cavities of the support elements.

### Example 19

The automatic retention system of any preceding example, wherein a frame is included that comprises an upper portion mounted to an upper part of a limb of the one or more limbs; and a lower portion mounted to a lower part of the limb; wherein the upper and lower parts of the limb are coupled together by a joint; wherein at least one upper retention member of a retention apparatus is mounted to the upper portion and surrounds at least a portion of the upper part of the limb; and wherein at least one other retention member of another separate retention apparatus is mounted to the lower portion and surrounds at least a portion of the lower part of the limb.

### Example 20

The automatic retention system of any preceding example, wherein the at least one control circuit includes multiple individual control circuits, and wherein the multiple automatic retention apparatuses are separately responsive to one or more separate control circuits of the multiple individual control circuits.

### Example 21

The automatic retention system of any preceding example, wherein separate control circuits are in communication with and responsive to at least one other control circuit of the multiple individual control circuits.

### Example 22

The automatic retention system of any preceding example, wherein the at least one sensor includes multiple individual sensors, and wherein the multiple individual control circuits are separately responsive to one or more sensors of the multiple individual sensors.

### Example 23

The automatic retention system of any preceding example, wherein the at least one sensor includes a blood pressure sensor, a temperature sensor configured to determine a temperature of the limb, a heart rate sensor, a temperature sensor configured to determine the ambient temperature around the limb, an accelerometer, an Inertial Measurement Unit (IMU), or sensors that measure oxygenation, respiratory rate, perspiration, brain function at a brain-machine interface, make cognitive function assessments, or any combination thereof.

### Example 24

The automatic retention system of any preceding example, wherein the at least one control circuit is a single control circuit operatively coupled to the multiple automatic retention apparatuses.

### Example 25

The automatic retention system of any preceding example, wherein the at least one sensor includes a first sensor mounted to a retention member of the multiple automatic retention apparatuses that is configured to generate input based on tension in the retention member, and a second sensor mounted to an object the retention member at least partially surrounds, and wherein the at least one sensor is configured to generate input based on movement of the object.

### Example 26

The automatic retention system of any preceding example, the system also including a cable inside a conduit, wherein the cable is coupled adjacent a first end to an actuator of at least one of the multiple automatic retention apparatuses; a cable actuator responsive to a control circuit that is coupled to the cable adjacent a second end; wherein the cable is selectively movable within the conduit according to movement of the cable actuator, and wherein the actuator is configured to adjust tension applied by the retention member according to movement of the cable relative to the conduit.

### Example 27

The automatic retention system of any preceding example, wherein the system includes a cable actuator that includes an electric motor mechanically coupled to a rotating member, the electric motor responsive to control input from the at least one control circuit; wherein the at least one control circuit is programmed to control the electric motor to rotate in a first direction and a second direction opposite the first direction to adjust the position of the cable relative to the conduit.

### Example 28

The automatic retention system of any preceding example, wherein the system includes a conduit that is optionally anchored at a first end to a first cable mount of the at least one automatic retention apparatus, and wherein the conduit is anchored at a second end to a cable second mount of a cable actuator.

### Example 29

The automatic retention system of any preceding example, the system also including a garment configured to surround at least a portion of one of the one or more limbs, wherein the garment defines an interior passageway and a cable and conduit are positioned inside the interior passageway.

### Example 30

The automatic retention system of any preceding example, the system also including multiple cables separately positioned inside individual conduits, the multiple cables extending between separate actuators of the multiple automatic retention apparatuses and the at least one control circuit; multiple cable actuators responsive to the control circuit that are each individually coupled at a first end of each of the multiple cables; wherein the multiple cables are selectively movable within the individual conduits according to movement of cable actuators, and wherein the separate actuators are coupled to a second end of each of the multiple cables; and wherein the separate actuators are configured to adjust tension applied by corresponding retention members according to movement of the individual cables relative to the individual conduits.

### Example 31

The automatic retention system of any preceding example, wherein the actuator of at least one of the multiple automatic retention apparatuses comprises a rotating member with multiple teeth engaging one or more recesses defined by the retention member, wherein the rotating member is rotatable in a first direction to reduce tension of the retention member, and wherein the rotating member is rotatable in a second direction opposite the first direction to increase tension of the retention member; wherein the actuator is mechanically coupled to the rotating member, wherein the actuator is arranged and configured to rotate the rotating member in the first direction and the second directions to increase or decrease tension of the retention member in response to input from the control circuit.

### Example 32

The automatic retention system of any preceding example, wherein the retention member is an elongate retention member, wherein one or more projections and recesses are defined in a portion of the retention member that is wider than it is thick, and narrower than it is long.

### Example 33

The automatic retention system of any preceding example, wherein at least one of the one or more recess and projections include through holes interspersed along the retention member.

### Example 34

The automatic retention system of any preceding example, wherein a rotating member rotates around an axis of rotation that is substantially parallel to a longitudinal axis defined by the retention member.

### Example 35

The automatic retention system of any preceding example, wherein a rotating member rotates around an axis of rotation that is substantially perpendicular to a longitudinal axis defined by the retention member.

### Example 36

The automatic retention system of any preceding example, wherein rotating a rotating member in a first direction displaces a first portion of the retention member relative to a second portion of the retention member to adjust tension of the retention member.

### Example 37

The automatic retention system of any preceding example, wherein the actuator of at least one of the multiple automatic retention apparatuses comprises an electric motor mechanically coupled to a rotating member, the electric motor responsive to control input from the at least one control circuit, wherein the at least one control circuit is programmed to control the electric motor to rotate in the first and second direction to adjust tension of the retention member.

### Example 38

The automatic retention system of any preceding example, wherein the actuator of at least one of the multiple automatic retention apparatuses comprises a rotating member engaging with at least a portion of the retention member, wherein the rotating member is rotatable in a first direction to reduce tension of the retention member, and wherein the rotating member is rotatable in a second direction opposite the first direction to increase tension of the retention member; wherein the actuator is mechanically coupled to the rotating member, wherein the actuator is arranged and configured to rotate the rotating member in the first direction and the second directions to increase or decrease tension of the retention member in response to input from the control circuit.

### Example 39

The automatic retention system of any preceding example, wherein a portion of the retention member engages the rotating member and is arranged and configured to wrap around the rotating member as it rotates.

### Example 40

The automatic retention system of any preceding example, wherein a control circuit of the at least one control circuits is configured to control the actuators according to values for one or more operating parameters, and criteria for one or more rules.

### Example 41

The automatic retention system of any preceding example, wherein the control circuit is configured to receive one or more values for operating parameters from a remote computing device via a communications link.

### Example 42

The automatic retention system of any preceding example, wherein the control circuit is configured to receive criteria for one or more rules from a remote computing device via a communications link.

### Example 43

The automatic retention system of any preceding example, wherein the control circuit is configured to automatically determine and update at least one value for one or more operating parameters and at least one criteria for one or more rules.

### Example 44

The automatic retention system of any preceding example, wherein the control circuit includes a memory, and wherein the control circuit is configured to retain an operational history of operating parameters including a first value of an operating parameter retained in the memory at a first time, and a second value of the operating parameter retained in the memory at a second later time, and wherein the first and second values are used to determine a third new value for the operating parameter.

### Example 45

The automatic retention system of any preceding example, wherein an operational history of an automatic retention apparatus is sent to a remote computer via a communication link.

### Example 46

The automatic retention system of any preceding example, wherein the actuator of at least one of the multiple automatic retention apparatuses comprises: a manual control mechanically coupled to a rotating member, the manual control arranged and configured to rotate the rotating member in a first and second direction to adjust tension of the retention member based on input from a user.

### Example 47

The automatic retention system of any preceding example, the system also including one or more inflatable cavities positioned between the retention member and the at least one limb, wherein the actuator is arranged and configured to inflate the one or more inflatable cavities to adjust pressure applied to the at least one limb by the retention member.

### Example 48

An automatic retention system for applying tension to one or more limbs, the system also including multiple automatic retention apparatuses that include a retention member surrounding a portion of at least one limb of the one or more limbs; one or more inflatable cavities positioned between the retention member and the at least one limb; and an actuator arranged and configured to inflate the one or more inflatable cavities to adjust pressure applied to the at least one limb by the retention member; at least one sensor arranged and configured to sense changes in a sense parameter associated with the one or more limbs; and at least one control circuit responsive to the at least one sensor, wherein the at least one control circuit is configured to control the actuators of the multiple retention apparatuses to adjust inflation of the one or more inflatable cavities according to input received from the at least one sensor.

### Example 49

The automatic retention system of any preceding example, wherein an automatic retention apparatus includes one or more inflatable cavities such as first and second cavities of the one or more inflatable cavities that are separate and distinct, and wherein the actuator is configured to selectively inflate the first cavity at a first pressure, and the second cavity at a second pressure that is different than the first pressure.

### Example 50

The automatic retention system of any preceding example, wherein the system includes an automatic retention apparatus with one or more inflatable cavities that are in fluid communication with each other.

### Example 51

The automatic retention system of any preceding example, wherein the system includes an automatic retention apparatus with one or more inflatable cavities that are arranged circumferentially around the retention member.

### Example 52

The automatic retention system of any preceding example, the system also including a fluid compression apparatus in fluid communication with one or more of inflatable cavities of an automatic retention apparatus; wherein the fluid compression apparatus is arranged and configured to introduce a fluid into the cavities to inflate the one or more inflatable cavities.

### Example 53

The automatic retention system of any preceding example, the system also including a fluid compression apparatus that includes an air compressor responsive to the at least one control circuit, and wherein the fluid compressed is air.

### Example 54

The automatic retention system of any preceding example, the system also including a fluid compression apparatus that includes a squeeze bulb, and wherein the fluid is air.

### Example 55

The automatic retention system of any preceding example, the system also including a one way valve configured to open when the compression apparatus introduces fluid into the cavities, and to otherwise automatically close to retain the fluid in the cavities.

### Example 56

The automatic retention system of any preceding example, wherein the at least one sensor includes a first sensor mounted to a retention member of the multiple automatic retention apparatus that is configured to generate input based on an interior pressure of at least one inflatable cavity, and a second sensor mounted to an object the retention member at least partially surrounds.

### Glossary of Definitions and Alternatives

While examples are illustrated in the drawings and described herein, this disclosure is to be considered as illustrative and not restrictive in character. The present disclosure is exemplary in nature and all changes, equivalents, and modifications that come within the spirit of the inventions as defined in the claims are included. The detailed description is included herein to discuss aspects of the examples illustrated in the drawings for the purpose of promoting an understanding of the principles of the inventions. No limitation of the scope of the inventions is thereby intended. Any alterations and further modifications in the described examples, and any further applications of the principles described herein are contemplated as would normally occur to one skilled in the art to which the inventions relate. Some examples are disclosed in detail, however some features that may not be relevant may have been left out for the sake of clarity.

Where there are references to publications, patents, and patent applications cited herein, they are understood to be incorporated by reference as if each individual publication, patent, or patent application were specifically and individually indicated to be incorporated by reference and set forth in its entirety herein.

Singular forms “a”, “an”, “the”, and the like include plural referents unless expressly discussed otherwise. As an illustration, references to “a device” or “the device” include one or more of such devices and equivalents thereof.

Directional terms, such as “up”, “down”, “top” “bottom”, “fore”, “aft”, “lateral”, “longitudinal”, “radial”, “circumferential”, etc., are used herein solely for the convenience of the reader in order to aid in the reader's understanding of the illustrated examples. The use of these directional terms does not in any manner limit the described, illustrated, and/or claimed features to a specific direction and/or orientation.

Multiple related items may be illustrated in the drawings with the same part number but differentiated by a letter for separate individual instances. These may be referred to generally by a distinguishable portion of the full name, and/or by the number alone. For example, if multiple “laterally extending elements” 90A, 90B, 90C, and 90D are illustrated in the drawings, the disclosure may refer to these as “laterally extending elements 90A-90D,” or as “laterally extending elements 90,” or by a distinguishable portion of the full name such as “elements 90”.

The language used in the disclosure are presumed to have only their plain and ordinary meaning, except as explicitly defined below. The words used in the definitions included herein are to only have their plain and ordinary meaning. Such plain and ordinary meaning is inclusive of all consistent dictionary definitions from the most recently published Webster's and Random House dictionaries. As used herein, the following definitions apply to the following terms or to common variations thereof (e.g., singular/plural forms, past/present tenses, etc.):

“About” with reference to numerical values generally refers to plus or minus 10% of the stated value. For example, if the stated value is 4.375, then use of the term “about 4.375” generally means a range between 3.9375 and 4.8125.

“Activate” generally is synonymous with “providing power to”, or refers to “enabling a specific function” of a circuit or electronic device that already has power.

“Actuator” generally refers to a device for activating or controlling the actions of an actuated device. This may include, but is not limited to, moving or controlling movement. An actuator may be an element or aspect of the actuated device, such as in the case of a valve that includes an actuator for opening and closing the valve. An actuator may actuate operation of the device by direct mechanical linkage, by signals sent to the device via electromagnetic energy traveling over a wire, optical fiber, or through the air, or by actuating an intervening apparatus that causes the desired actuation of the target device.

“And/or” is inclusive here, meaning “and” as well as “or”. For example, “P and/or Q” encompasses, P, Q, and P with Q; and, such “P and/or Q” may include other elements as well.

“Antenna” or “Antenna system” generally refers to an electrical device, or series of devices, in any suitable configuration, that converts electric power into electromagnetic radiation. Such radiation may be either vertically, horizontally, or circularly polarized at any frequency along the electromagnetic spectrum. Antennas transmitting with circular polarity may have either right-handed or left-handed polarization.

In the case of radio waves, an antenna may transmit at frequencies ranging along electromagnetic spectrum from extremely low frequency (ELF) to extremely high frequency (EHF). An antenna or antenna system designed to transmit radio waves may comprise an arrangement of metallic conductors (elements), electrically connected (often through a transmission line) to a receiver or transmitter. An oscillating current of electrons forced through the antenna by a transmitter can create an oscillating magnetic field around the antenna elements, while the charge of the electrons also creates an oscillating electric field along the elements. These time-varying fields radiate away from the antenna into space as a moving transverse electromagnetic field wave. Conversely, during reception, the oscillating electric and magnetic fields of an incoming electromagnetic wave exert force on the electrons in the antenna elements, causing them to move back and forth, creating oscillating currents in the antenna. These currents can then be detected by receivers and processed to retrieve digital or analog signals or data.

Antennas can be designed to transmit and receive radio waves substantially equally in all horizontal directions (omnidirectional antennas), or preferentially in a particular direction (directional or high gain antennas). In the latter case, an antenna may also include additional elements or surfaces which may or may not have any physical electrical connection to the transmitter or receiver. For example, parasitic elements, parabolic reflectors or horns, and other such non-energized elements serve to direct the radio waves into a beam or other desired radiation pattern. Thus antennas may be configured to exhibit increased or decreased directionality or “gain” by the placement of these various surfaces or elements. High gain antennas can be configured to direct a substantially large portion of the radiated electromagnetic energy in a given direction that may be vertical horizontal or any combination thereof.

Antennas may also be configured to radiate electromagnetic energy within a specific range of vertical angles (i.e. “takeoff angles) relative to the earth in order to focus electromagnetic energy toward an upper layer of the atmosphere such as the ionosphere. By directing electromagnetic energy toward the upper atmosphere at a specific angle, specific skip distances may be achieved at particular times of day by transmitting electromagnetic energy at particular frequencies.

Other examples of antennas include emitters and sensors that convert electrical energy into pulses of electromagnetic energy in the visible or invisible light portion of the electromagnetic spectrum. Examples include light emitting diodes, lasers, and the like that are configured to generate electromagnetic energy at frequencies ranging along the electromagnetic spectrum from far infrared to extreme ultraviolet.

“Appendage” or “Limb” generally refers to any portion of the human or animal body. Examples include neck, arm, leg, finger, torso, head, foot etc.

“Battery” generally refers to an electrical energy storage device or storage system including multiple energy storage devices. A battery may include one or more separate electrochemical cells, each converting stored chemical energy into electrical energy by a chemical reaction to generate an electromotive force (or “EMF” measured in Volts). An individual battery cell may have a positive terminal (cathode) with a higher electrical potential, and a negative terminal (anode) that is at a lower electrical potential than the cathode. Any suitable electrochemical cell may be used that employ any suitable chemical process, including galvanic cells, electrolytic cells, fuel cells, flow cells and voltaic piles. When a battery is connected to an external circuit, electrolytes are able to move as ions within the battery, allowing the chemical reactions to be completed at the separate terminals thus delivering energy to the external circuit.

A battery may be a “primary” battery that can produce current immediately upon assembly. Examples of this type include alkaline batteries, nickel oxyhydroxide, lithium-copper, lithium-manganese, lithium-iron, lithium-carbon, lithium-thionyl chloride, mercury oxide, magnesium, zinc-air, zinc-chloride, or zinc-carbon batteries. Such batteries are often referred to as “disposable” insofar as they are generally not rechargeable and are discarded or recycled after discharge.

A battery may also be a “secondary” or “rechargeable” battery that can produce little or no current until charged. Examples of this type include lead-acid batteries, valve regulated lead-acid batteries, sealed gel-cell batteries, and various “dry cell” batteries such as nickel-cadmium (NiCd), nickel-zinc (NiZn), nickel metal hydride (NiMH), and lithium-ion (Li-ion) batteries.

“Braking mechanism” generally refers to a selectively engageable mechanism configured to reduce or halt the movement or rotation of one object with respect to another. In one example, a braking mechanism uses friction between two surfaces selectively pressed together to convert the kinetic energy of the moving or rotating object into heat, though other methods of energy conversion may be employed. Regenerative braking converts much of the energy to electrical energy, which may be stored for later use. Other methods convert kinetic energy into potential energy in such stored forms as pressurized air or pressurized oil. Eddy current brakes use magnetic fields to convert kinetic energy into electric current in the brake disc, fin, or rail, which is converted into heat. Still other braking methods even transform kinetic energy into different forms, for example by transferring the energy to a rotating flywheel.

Another example of a braking mechanism is a ratchet which allows continuous linear or rotary motion in only one direction while preventing motion in the opposite direction. A ratchet may include a series of engagement members such as teeth arranged around a gear or on linear rack. A pivoting, spring-loaded finger called a pawl engages the teeth. The teeth are uniform but asymmetrical, with each tooth having a moderate slope on one edge and a much steeper slope on the other edge. When the teeth are moving in the unrestricted (i.e., forward) direction, the pawl easily slides up and over the gently sloped edges of the teeth, with a biasing element such as a spring forcing it into the depression between the teeth as it passes the tip of each tooth. When the teeth attempt to move in the opposite (backward) direction the pawl catches on the steeply sloped edge of the first tooth it encounters, thereby locking it against the tooth and preventing any further motion in that direction until the pawl is released.

“Cable” generally refers to one or more elongate strands of material that has tensile strength, and/or compressive strength. In other words, a cable may be a relatively flexible elongate structure of one or more strands that tends to resist being pulled apart or stretched, and/or is generally able to resist being compressed together. Examples include wire rope, flexible shafts, Bowden cables, coaxial cable, twisted pair electrical wire, a single strand of wire, as well as non-wire ropes made of natural or synthetic fibers.

“Communication Link” generally refers to a connection between two or more communicating entities and may or may not include a communications channel between the communicating entities. The communication between the communicating entities may occur by any suitable means. For example, the connection may be implemented as an actual physical link, an electrical link, an electromagnetic link, a logical link, or any other suitable linkage facilitating communication.

In the case of an actual physical link, communication may occur by multiple components in the communication link configured to respond to one another by physical movement of one element in relation to another. In the case of an electrical link, the communication link may be composed of multiple electrical conductors electrically connected to form the communication link.

In the case of an electromagnetic link, the connection may be implemented by sending or receiving electromagnetic energy at any suitable frequency, thus allowing communications to pass as electromagnetic waves. These electromagnetic waves may or may not pass through a physical medium such as an optical fiber, or through free space, or any combination thereof. Electromagnetic waves may be passed at any suitable frequency including any frequency in the electromagnetic spectrum.

A communication link may include any suitable combination of hardware which may include software components as well. Such hardware may include routers, switches, networking endpoints, repeaters, signal strength enters, hubs, and the like.

In the case of a logical link, the communication link may be a conceptual linkage between the sender and recipient such as a transmission station in the receiving station. Logical link may include any combination of physical, electrical, electromagnetic, or other types of communication links.

“Computer” generally refers to any computing device configured to compute a result from any number of input values or variables. A computer may include a control circuit for performing calculations to process input or output. A computer may include a memory for storing values to be processed by the processor, or for storing the results of previous processing.

A computer may also be configured to accept input and output from a wide array of input and output devices for receiving or sending values. Such devices include other computers, keyboards, mice, visual displays, printers, industrial equipment, and systems or machinery of all types and sizes. For example, a computer can control a network or network interface to perform various network communications upon request. The network interface may be part of the computer, or characterized as separate and remote from the computer.

A computer may be a single, physical, computing device such as a desktop computer, a laptop computer, or may be composed of multiple devices of the same type such as a group of servers operating as one device in a networked cluster, or a heterogeneous combination of different computing devices operating as one computer and linked together by a communication network. The communication network connected to the computer may also be connected to a wider network such as the internet. Thus a computer may include one or more physical processors or other computing devices or circuitry, and may also include any suitable type of memory.

A computer may also be a virtual computing platform having an unknown or fluctuating number of physical processors and memories or memory devices. A computer may thus be physically located in one geographical location or physically spread across several widely scattered locations with multiple processors linked together by a communication network to operate as a single computer.

The concept of “computer” and “processor” within a computer or computing device also encompasses any such processor or computing device serving to make calculations or comparisons as part of the disclosed system. Processing operations related to threshold comparisons, rules comparisons, calculations, and the like occurring in a computer may occur, for example, on separate servers, the same server with separate processors, or on a virtual computing environment having an unknown number of physical processors as described above.

A computer may be optionally coupled to one or more visual displays and/or may include an integrated visual display. Likewise, displays may be of the same type, or a heterogeneous combination of different visual devices. A computer may also include one or more operator input devices such as a keyboard, mouse, touch screen, laser or infrared pointing device, or gyroscopic pointing device to name just a few representative examples. Also, besides a display, one or more other output devices may be included such as a printer, plotter, industrial manufacturing machine, 3D printer, and the like. As such, various display, input and output device arrangements are possible.

Multiple computers or computing devices may be configured to communicate with one another or with other devices over wired or wireless communication links to form a network. Network communications may pass through various computers operating as network appliances such as switches, routers, firewalls or other network devices or interfaces before passing over other larger computer networks such as the internet. Communications can also be passed over the network as wireless data transmissions carried over electromagnetic waves through transmission lines or free space. Such communications include using WiFi or other Wireless Local Area Network (WELAN) or a cellular transmitter/receiver to transfer data.

“Controller” or “control circuit” generally refers to a mechanical or electronic device configured to control the behavior of another mechanical or electronic device. A controller or “control circuit” is optionally configured to provide signals or other electrical impulses that may be received and interpreted by the controlled device to indicate how it should behave.

“Coupling device” generally refers to a device for coupling one object to another. Examples include a belt buckle, a zipper, a latch, a padlock, a trailer hitch, a clothing button, an electrical connector, boot bindings for a snow board or snow ski, and foot straps for a water ski, kite board, surf board, wave board, or sail board, to name a few non-limiting examples.

“Data” generally refers to one or more values of qualitative or quantitative variables that are usually the result of measurements. Data may be considered “atomic” as being finite individual units of specific information. Data can also be thought of as a value or set of values that includes a frame of reference indicating some meaning associated with the values. For example, the number “2” alone is a symbol that absent some context is meaningless. The number “2” may be considered “data” when it is understood to indicate, for example, the number of items produced in an hour.

Data may be organized and represented in a structured format. Examples include a tabular representation using rows and columns, a tree representation with a set of nodes considered to have a parent-children relationship, or a graph representation as a set of connected nodes to name a few.

The term “data” can refer to unprocessed data or “raw data” such as a collection of numbers, characters, or other symbols representing individual facts or opinions. Data may be collected by sensors in controlled or uncontrolled environments, or generated by observation, recording, or by processing of other data. The word “data” may be used in a plural or singular form. The older plural form “datum” may be used as well.

“Database” also referred to as a “data store”, “data repository”, or “knowledge base” generally refers to an organized collection of data. The data is typically organized to model aspects of the real world in a way that supports processes obtaining information about the world from the data. Access to the data is generally provided by a “Database Management System” (DBMS) consisting of an individual computer software program or organized set of software programs that allow user to interact with one or more databases providing access to data stored in the database (although user access restrictions may be put in place to limit access to some portion of the data).

In another aspect, the DBMS provides various functions that allow entry, storage and retrieval of large quantities of information as well as ways to manage how that information is organized. A database is not generally portable across different DBMSs, but different DBMSs can interoperate by using standardized protocols and languages such as Structured Query Language (SQL), Open Database Connectivity (ODBC), Java Database Connectivity (JDBC), or Extensible Markup Language (XML) to allow a single application to work with more than one DBMS.

In another aspect, a database may implement “smart contracts” which include rules written in computer code that automatically execute specific actions when predetermined conditions have been met and verified. Examples of such actions include, but are not limited to, releasing funds to the appropriate parties, registering a vehicle, sending notifications, issuing a certificate of ownership transfer, and the like. The database may then be updated when the transactions specified in the rules encoded in the smart contract are completely executed. In another aspect, the transaction specified in the rolls may be irreversible and automatically executed without the possibility of manual intervention. In another aspect, only parties specified in the rules of the smart contract who have been granted permission may be notified or allowed to see the results.

Databases and their corresponding database management systems are often classified according to a particular database model they support. Examples include a DBMS that relies on the “relational model” for storing data, usually referred to as Relational Database Management Systems (RDBMS). Such systems commonly use some variation of SQL to perform functions which include querying, formatting, administering, and updating an RDBMS. Other examples of database models include the “object” model, chained model (such as in the case of a “blockchain” database), the “object-relational” model, the “file”, “indexed file” or “flat-file” models, the “hierarchical” model, the “network” model, the “document” model, the “XML” model using some variation of XML, the “entity-attribute-value” model, and others.

Examples of commercially available database management systems include PostgreSQL provided by the PostgreSQL Global Development Group; Microsoft SQL Server provided by the Microsoft Corporation of Redmond, Washington, USA; MySQL and various versions of the Oracle DBMS, often referred to as simply “Oracle” both separately offered by the Oracle Corporation of Redwood City, California, USA; the DBMS generally referred to as “SAP” provided by SAP SE of Walldorf, Germany; and the DB2 DBMS provided by the International Business Machines Corporation (IBM) of Armonk, New York, USA.

The database and the DBMS software may also be referred to collectively as a “database”. Similarly, the term “database” may also collectively refer to the database, the corresponding DBMS software, and a physical computer or collection of computers. Thus the term “database” may refer to the data, software for managing the data, and/or a physical computer that includes some or all of the data and/or the software for managing the data.

“Electrically connected” generally refers to a configuration of two objects that allows electricity to flow between them or through them. In one example, two conductive materials are physically adjacent one another and are sufficiently close together so that electricity can pass between them. In another example, two conductive materials are in physical contact allowing electricity to flow between them.

“Gear” generally refers to a machine part having engagement teeth, or cogs, which extend outwardly away from the body of the gear. The teeth are configured to mesh with another part have corresponding similarly spaced teeth or similarly spaced holes that extend at least a portion of the way through the other part. Types of gears include spur, helical, skew, double helical, bevel, spiral bevel, hypoid, crown, worm, non-circular, rack and pinion, epicyclic, sun and planet, harmonic, cage, cycloidal, and magnetic to name a few nonlimiting examples.

Worm gears resemble screws and mesh with a worm wheel, which looks similar to a spur gear. Worm-and-gear sets are a simple and compact way to achieve a high torque, low speed gear ratio. A worm gear is a species of helical gear, but its helix angle is usually somewhat large (close to 90 degrees) and its body is usually fairly long in the axial direction. These attributes give it screw like qualities. The distinction between a worm and a helical gear is that at least one tooth persists for a full rotation around the helix. A worm gear may be thought of as having a single tooth in the case where the tooth persists for several turns around the helix. A worm gear may also be thought of as having more than one tooth when viewed perpendicular to the long axis of the gear. The reappearing tooth at intervals along the length of the worm may thus be thought of as multiple teeth.

In a worm-and-gear set, the worm can always drive the gear. However, if the gear attempts to drive the worm, it may or may not succeed. Particularly if the lead angle is small, the gears teeth may simply lock against the worm's teeth, because the force component circumferential to the worm is not sufficient to overcome friction. In traditional music boxes, however, the gear drives the worm, which has a large helix angle. A worm and gear set may be “self-locking”, as when it is desired to set the position of a mechanism by turning the worm and then have the mechanism hold that position without allowing retrograde rotation. An example is the machine head found on some types of stringed instruments.

“Hole” generally refers to a hollowed out area defined by a solid body or surface. A hole may extend into the solid body or surface without passing through such as in the case of an indention, depression, or pit. A hole may also pass through one side of an object to another side, thus passing completely through the object. The second side may be the same as the first, such as in the case of loop inside a solid body. Holes may have any suitable shape such as a circle, rectangle, oval, square, triangle, and the like.

“Identifier” generally refers to a name that identifies (that is, labels the identity of) either a unique thing or a unique class of things, where the “object” or class may be an idea, physical object (or class thereof), or physical substance (or class thereof). The abbreviation “ID” often refers to identity, identification (the process of identifying), or an identifier (that is, an instance of identification). An identifier may or may not include words, numbers, letters, symbols, shapes, colors, sounds, or any combination of those.

The words, numbers, letters, or symbols may follow an encoding system (wherein letters, digits, words, or symbols represent ideas or longer identifiers) or they may simply be arbitrary. When an identifier follows an encoding system, it is often referred to as a code or ID code. Identifiers that do not follow any encoding scheme are often said to be arbitrary IDs because they are arbitrarily assigned without meaning in any other context beyond identifying something.

“Input” generally refers to something put in, such as a physical substance put in (e.g. increased input of fuel), power or energy put into a machine or system usually with the intent of sizable recovery in the form of output, a component of production (such as land, labor, or raw materials), signals, data, or information fed into a computer, advice or comment, or a stimulus that acts on and is integrated into a bodily system. In the case of information fed into a computer, the input may be generated by a sensor detecting a sense parameter. In this instances, the time-varying values of the sense parameter are at least part of the input.

“Memory” generally refers to any storage system or device configured to retain data or information. Each memory may include one or more types of solid-state electronic memory, magnetic memory, or optical memory, just to name a few. Memory may use any suitable storage technology, or combination of storage technologies, and may be volatile, nonvolatile, or a hybrid combination of volatile and nonvolatile varieties. By way of nonlimiting example, each memory may include solid-state electronic Random Access Memory (RAM), Sequentially Accessible Memory (SAM) (such as the First-In, First-Out (FIFO) variety or the Last-In-First-Out (LIFO) variety), Programmable Read Only Memory (PROM), Electronically Programmable Read Only Memory (EPROM), or Electrically Erasable Programmable Read Only Memory (EEPROM).

Memory can refer to Dynamic Random Access Memory (DRAM) or any variants, including static random access memory (SRAM), Burst SRAM or Synch Burst SRAM (BSRAM), Fast Page Mode DRAM (FPM DRAM), Enhanced DRAM (EDRAM), Extended Data Output RAM (EDO RAM), Extended Data Output DRAM (EDO DRAM), Burst Extended Data Output DRAM (REDO DRAM), Single Data Rate Synchronous DRAM (SDR SDRAM), Double Data Rate SDRAM (DDR SDRAM), Direct Rambus DRAM (DRDRAM), or Extreme Data Rate DRAM (XDR DRAM).

Memory can also refer to non-volatile storage technologies such as non-volatile read access memory (NVRAM), flash memory, non-volatile static RAM (nvSRAM), Ferroelectric RAM (FeRAM), Magnetoresistive RAM (MRAM), Phase-change memory (PRAM), conductive-bridging RAM (CBRAM), Silicon-Oxide-Nitride-Oxide-Silicon (SONOS), Resistive RAM (RRAM), Domain Wall Memory (DWM) or “Racetrack” memory, Nano-RAM (NRAM), or Millipede memory. Other non-volatile types of memory include optical disc memory (such as a DVD or CD ROM), a magnetically encoded hard disc or hard disc platter, floppy disc, tape, or cartridge media. The concept of a “memory” includes the use of any suitable storage technology or any combination of storage technologies.

“Motor” generally refers to a rotating machine that transforms electrical or chemical energy into mechanical energy, such as by a rotating shaft. Examples include electric motors and internal combustion engines.

“Movement” generally refers to an act of changing physical a physical property such as position, dimension, posture, angle of incidence, or location to name a few nonlimiting examples. Movement of an object may be caused by the object, by the activities of other objects acting on the object either directly or indirectly, and/or by the actions of environmental forces such as gravity, wind, and the like.

“Multiple” as used herein is synonymous with the term “plurality” and refers to more than one, or by extension, two or more.

“Network” or “Computer Network” generally refers to a telecommunications network that allows computers to exchange data. Computers can pass data to each other along data connections by transforming data into a collection of datagrams or packets. The connections between computers and the network may be established using either cables, optical fibers, or via electromagnetic transmissions such as for wireless network devices.

Computers coupled to a network may be referred to as “nodes” or as “hosts” and may originate, broadcast, route, or accept data from the network. Nodes can include any computing device such as personal computers, phones, servers as well as specialized computers that operate to maintain the flow of data across the network, referred to as “network devices”. Two nodes can be considered “networked together” when one device is able to exchange information with another device, whether or not they have a direct connection to each other.

Examples of wired network connections may include Digital Subscriber Lines (DSL), coaxial cable lines, or optical fiber lines. The wireless connections may include BLUETOOTH, Worldwide Interoperability for Microwave Access (WiMAX), infrared channel or satellite band, or any wireless local area network (Wi-Fi) such as those implemented using the Institute of Electrical and Electronics Engineers' (IEEE) 802.11 standards (e.g. 802.11(a), 802.11(b), 802.11(g), or 802.11(n) to name a few). Wireless links may also include or use any cellular network standards used to communicate among mobile devices including 1G, 2G, 3G, or 4G. The network standards may qualify as 1G, 2G, etc. by fulfilling a specification or standards such as the specifications maintained by International Telecommunication Union (ITU). For example, a network may be referred to as a “3G network” if it meets the criteria in the international Mobile Telecommunications-2000 (IMT-2000) specification regardless of what it may otherwise be referred to. A network may be referred to as a “4G network” if it meets the requirements of the International Mobile Telecommunications Advanced (IMTAdvanced) specification. Examples of cellular network or other wireless standards include AMPS, GSM, GPRS, UMTS, LTE, LTE Advanced, Mobile WiMAX, and WiMAX-Advanced.

Cellular network standards may use various channel access methods such as FDMA, TDMA, CDMA, or SDMA. Different types of data may be transmitted via different links and standards, or the same types of data may be transmitted via different links and standards.

The geographical scope of the network may vary widely. Examples include a body area network (BAN), a personal area network (PAN), a low power wireless Personal Area Network using IPv6 (6LoWPAN), a local-area network (LAN), a metropolitan area network (MAN), a wide area network (WAN), or the Internet.

A network may have any suitable network topology defining the number and use of the network connections. The network topology may be of any suitable form and may include point-to-point, bus, star, ring, mesh, or tree. A network may be an overlay network which is virtual and is configured as one or more layers that use or “lay on top of” other networks.

A network may utilize different communication protocols or messaging techniques including layers or stacks of protocols. Examples include the Ethernet protocol, the internet protocol suite (TCP/IP), the ATM (Asynchronous Transfer Mode) technique, the SONET (Synchronous Optical Networking) protocol, or the SDH (Synchronous Digital Hierarchy) protocol. The TCP/IP internet protocol suite may include application layer, transport layer, internet layer (including, e.g., IPv6), or the link layer.

“Optionally” as used herein means discretionary; not required; possible, but not compulsory; left to personal choice.

“Personal computing device” generally refers to a computing device configured for use by individual people. Examples include mobile devices such as Personal Digital Assistants (PDAs), tablet computers, wearable computers installed in items worn on the human body such as in eye glasses, laptop computers, portable music/video players, computers in automobiles, or cellular telephones such as smart phones. Personal computing devices can be devices that are typically not mobile such as desk top computers, game consoles, or server computers. Personal computing devices may include any suitable input/output devices and may be configured to access a network such as through a wireless or wired connection, and/or via other network hardware.

“Predominately” as used herein is synonymous with greater than 50%.

“Processor” generally refers to one or more electronic components configured to operate as a single unit configured or programmed to process input to generate an output. Alternatively, when of a multi-component form, a processor may have one or more components located remotely relative to the others. One or more components of each processor may be of the electronic variety defining digital circuitry, analog circuitry, or both.

In one example, each processor is of a conventional, integrated circuit microprocessor arrangement, such as one or more PENTIUM, i3, i5 or i7 processors supplied by INTEL Corporation of Santa Clara, California, USA. Other examples of commercially available processors include but are not limited to the X8 and Freescale Coldfire processors made by Motorola Corporation of Schaumburg, Illinois, USA; the ARM processor and TEGRA System on a Chip (SoC) processors manufactured by Nvidia of Santa Clara, California, USA; the POWER7 processor manufactured by International Business Machines of White Plains, New York, USA; any of the FX, Phenom, Athlon, Sempron, or Opteron processors manufactured by Advanced Micro Devices of Sunnyvale, California, USA; or the Snapdragon SoC processors manufactured by Qalcomm of San Diego, California, USA.

A processor also includes Application-Specific Integrated Circuit (ASIC). An ASIC is an Integrated Circuit (IC) customized to perform a specific series of logical operations is controlling a computer to perform specific tasks or functions. An ASIC is an example of a processor for a special purpose computer, rather than a processor configured for general-purpose use. An application-specific integrated circuit generally is not reprogrammable to perform other functions and may be programmed once when it is manufactured.

In another example, a processor may be of the “field programmable” type. Such processors may be programmed multiple times “in the field” to perform various specialized or general functions after they are manufactured. A field-programmable processor may include a Field-Programmable Gate Array (FPGA) in an integrated circuit in the processor. FPGA may be programmed to perform a specific series of instructions which may be retained in nonvolatile memory cells in the FPGA. The FPGA may be configured by a customer or a designer using a hardware description language (IDL). In FPGA may be reprogrammed using another computer to reconfigure the FPGA to implement a new set of commands or operating instructions. Such an operation may be executed in any suitable means such as by a firmware upgrade to the processor circuitry.

Just as the concept of a computer is not limited to a single physical device in a single location, so also the concept of a “processor” is not limited to a single physical logic circuit or package of circuits but includes one or more such circuits or circuit packages possibly contained within or across multiple computers in numerous physical locations. In a virtual computing environment, an unknown number of physical processors may be actively processing data, the unknown number may automatically change over time as well.

The concept of a “processor” includes a device configured or programmed to make threshold comparisons, rules comparisons, calculations, or perform logical operations applying a rule to data yielding a logical result (e.g. “true” or “false”). Processing activities may occur in multiple single processors on separate servers, on multiple processors in a single server with separate processors, or on multiple processors physically remote from one another in separate computing devices.

“Portion” means a part of a whole, either separated from or integrated with it.

“Remote” generally refers to physical separation or interval. The term may as used herein does not naturally imply a larger than usual physical separation. A separation of less than 10 feet may be thought of as “remote”, as would a separation of greater than 10 feet, greater than 10,000 miles, or greater than a light year.

“Retention Member” generally refers to an element, component, part, piece, or assembly configured to retain a first object in relation to a second object, or to generally apply tension or pressure to an object. The second object may be the retention member itself such as in the case of a retention member whose purpose is to hold itself in position relative to the first object. A retention member may be an assembly of multiple interrelated members that together operate as a retention member such as multiple interrelated segments, strands, or other elements intertwined or otherwise coupled together.

Examples of retention members include, but are not limited to, elongate structures such as a strap, chain, cable, wire, belt, string, and the like. A retention member may include coupling devices such as a snap, latch, coupler, clasp, or hook. Other examples include fasteners such as a screw, bolt, nail, brad, nut, or staple.

“Rule” generally refers to a conditional statement with at least two outcomes. A rule may be compared to available data which can yield a positive result (all aspects of the conditional statement of the rule are satisfied by the data), or a negative result (at least one aspect of the conditional statement of the rule is not satisfied by the data). One example of a rule is shown below as pseudo code of an “if/then/else” statement that may be coded in a programming language and executed by a processor in a computer

```
if(clouds.areGrey( ) and
(clouds.numberOfClouds > 100)) then
{
  prepare for rain;
} else {
  Prepare for sunshine;
}
```

“Sense parameter” generally refers to a property of the environment detectable by a sensor. As used herein, sense parameter can be synonymous with an operating condition, environmental factor, sensor parameter, or environmental condition. Sense parameters may include temperature, air pressure, speed, acceleration, tension, weight, force, angle of defection of an object with respect to another object or with respect to gravity, the presence or intensity of sound or light or other electromagnetic phenomenon, the strength and/or orientation of a magnetic or electrical field, and the like. Other examples include, hear rate, changes in location according to a location service such as the Global Positioning System (GPS), blood pressure, and the like.

“Sensor” generally refers to a transducer configured to sense or detect a characteristic of the environment local to the sensor. For example, sensors may be constructed to detect events or changes in quantities or sense parameters providing a corresponding output, generally as an electrical or electromagnetic signal. A sensor's sensitivity indicates how much the sensor's output changes when the input quantity being measured changes.

“Signal” generally refers to a function or means of representing information. It may be thought of as the output of a transformation or encoding process. The concept generally includes a change in the state of a medium or carrier that conveys the information. The medium can be any suitable medium such as air, water, electricity, magnetism, or electromagnetic energy such as in the case of radio waves, pulses of visible or invisible light, and the like.

As used herein, a “signal” implies a representation of meaningful information. Arbitrary or random changes in the state of a carrier medium are generally not considered “signals” and may be considered “noise”. For example, arbitrary binary data streams are not considered as signals. On the other hand, analog and digital signals that are representations of analog physical quantities are examples of signals. A signal is commonly not useful without some way to transmit or send the information, and a receiver responsive to the transmitter for receiving the information.

In a communication system, for example, a transmitter encodes a message to a signal, which is carried to a receiver by the communications channel. For example, the words “The time is 12 o'clock” might be the message spoken into a telephone. The telephone transmitter may then convert the sounds into an electrical voltage signal. The signal is transmitted to the receiving telephone by wires, at the receiver it is reconverted into sounds.

Signals may be thought of as “discrete” or “continuous.” Discrete-time signals are often referred to as time series in other fields. Continuous-time signals are often referred to as continuous signals even when the signal functions are not continuous, such as in a square-wave signal.

Another categorization is signals which are “discrete-valued” and “continuous-valued”. Particularly in digital signal processing a digital signal is sometimes defined as a sequence of discrete values, that may or may not be derived from an underlying continuous-valued physical process. In other contexts, digital signals are defined as the continuous-time waveform signals in a digital system, representing a bit-stream. In the first case, a signal that is generated by means of a digital modulation method may be considered as converted to an analog signal, while it may be considered as a digital signal in the second case.

“Surround” as used herein means to “extend around at least a portion of.” Implicit is a physical or conceptual perimeter around an object that is at least partially enclosed by another object, or arrangement of multiple objects. This includes to fully envelope, to enclose on all sides, and/or to extend fully around the margin or edge. The term may also contemplates intermittent spacing between placement of objects around a portion of another object, such as in the case of chairs that are said to surround a table, or police officers surrounding a building. The term also may be used in the abstract such as when a person's activities are surrounded by secrecy.

“Triggering a Rule” generally refers to an outcome that follows when all elements of a conditional statement expressed in a rule are satisfied. In this context, a conditional statement may result in either a positive result (all conditions of the rule are satisfied by the data), or a negative result (at least one of the conditions of the rule is not satisfied by the data) when compared to available data. The conditions expressed in the rule are triggered if all conditions are met causing program execution to proceed along a different path than if the rule is not triggered.

What is claimed is:

1. An automatic retention system for applying pressure to one or more limbs, comprising:
multiple automatic retention apparatuses that include:
a retention member surrounding a portion of at least one limb of the one or more limbs; and
an actuator arranged and configured to engage the retention member, wherein the actuator is configured to actuate the retention member to adjust pressure applied to the at least one limb by the retention member:
at least one sensor arranged and configured to sense changes in a sense parameter associated with the one or more limbs; and
at least one control circuit responsive to the at least one sensor, wherein the at least one control circuit is configured to control the actuators of the multiple retention apparatuses to adjust pressure applied to the at least one of the one or more limbs according to input received from the at least one sensor.

2. The automatic retention system of claim 1, comprising:
a garment configured to surround at least a portion of one of the one or more limbs.

3. The automatic retention system of claim 2, wherein at least one of the multiple automatic retention apparatuses is positioned outside the garment.

4. The automatic retention system of claim 2, wherein at least one of the multiple automatic retention apparatuses is positioned within a cavity defined within the garment.

5. The automatic retention system of claim 2, wherein a retention member of at least one of the multiple automatic retention apparatuses is woven into the garment.

6. The automatic retention system of claim 5, wherein the garment includes an actuator mount configured to couple an actuator of at least one of the multiple automatic retention apparatuses adjacent to the retention member.

7. The automatic retention system of claim 2, wherein a retention member of at least one of the multiple automatic retention apparatuses includes a cloth strap that is mounted to the garment at a predetermined mount location.

8. The automatic retention system of claim 7, wherein the cloth strap is mounted inside the garment, wherein the garment defines an opening through Which the actuator engages the retention member to adjust tension on the cloth strap.

9. The automatic retention system of claim 2, wherein the garment defines an interior passageway and at least a portion of one retention member of the multiple automatic retention apparatuses is positioned inside the interior passageway

10. The automatic retention system of claim 9, wherein the actuator of the at least one retention member is mounted outside the interior passageway.

11. The automatic retention system of claim 2, wherein the garment includes multiple mounts corresponding to each of the multiple automatic retention apparatuses.

12. The automatic retention system of claim 1, wherein at least one automatic retention apparatus of the multiple automatic retention apparatuses is mounted remote from the at least one control circuit.

13. The automatic retention system of claim 1, wherein at least one automatic retention apparatus of the multiple automatic retention apparatuses is mounted to a housing and the at least one control circuit is mounted inside the housing.

14. The automatic retention system of claim 1, wherein an upper retention apparatus of the at least one automatic retention apparatuses is mounted at an upstream location of a limb of the one or more limbs, and a separate lower retention apparatus of the at least one automatic retention apparatuses is mounted at a downstream location of the limb.

15. The automatic retention system of claim 14, wherein the upstream and downstream portions of the limb are coupled together by a joint of the limb.

16. The automatic retention system of claim 1, comprising:
a frame configured to receive at least a portion of a limb of the one or more limbs, wherein at least one of the multiple retention members is mounted to the frame.

17. The automatic retention system of claim 16, wherein the frame includes two frame members on opposing sides of the limb, and wherein the two frame members are longitudinally aligned with a reference plane; and

18. The automatic retention system of claim 16, comprising:
multiple support elements coupled together and coupled to the frame, the multiple support elements also aligned with the reference plane;
wherein the multiple support elements and the frame are coupled together and rotatable substantially parallel to the reference plane, and are prevented from rotating away from the reference plane by virtue of multiple projecting members positioned within corresponding cavities of the support elements.

19. The automatic retention system of claim 16, wherein the frame comprises:
an upper portion mounted to an upper part of a limb of the one or more limbs; and
a lower portion mounted to a lower part of the limb;
wherein the upper and lower parts of the limb are coupled together by a joint;
wherein at least one upper retention member of a retention apparatus is mounted to the upper portion and surrounds at least a portion of the upper part of the limb; and
wherein at least one other retention member of another separate retention apparatus is mounted to the lower portion and surrounds at least a portion of the lower part of the limb.

20. The automatic retention system of claim 1, wherein the at least one control circuit includes multiple individual control circuits, and wherein the multiple automatic retention apparatuses are separately responsive to one or more separate control circuits of the multiple individual control circuits.

21. The automatic retention system of claim 20, wherein the separate control circuits are in communication with and responsive to at least one other control circuit of the multiple individual control circuits.

22. The automatic retention system of claim 1, wherein the at least one sensor includes multiple individual sensors, and wherein the multiple individual control circuits are separately responsive to one or more sensors of the multiple individual sensors.

23. The automatic retention system of claim 1, wherein the at least one sensor includes a blood pressure sensor, a temperature sensor configured to determine a temperature of the limb, a heart rate sensor, a temperature sensor configured to determine the ambient temperature around the limb, an accelerometer, Inertial Measurement Unit (IMU), or any combination thereof.

24. The automatic retention system of claim 1, wherein the at least one control circuit is a single control circuit operatively coupled to the multiple automatic retention apparatuses.

25. The automatic retention system of claim 1, wherein the at least one sensor includes a first sensor mounted to a retention member of the multiple automatic retention apparatus that is configured to generate input based on tension in the retention member, and a second sensor mounted to an object the retention member at least partially surrounds, and the at least one sensor is configured to generate input based on movement of the object.

26. The automatic retention system of claim 1, comprising:
a cable inside a conduit, wherein the cable is coupled adjacent a first end to an actuator of at least one of the multiple automatic retention apparatuses;
a cable actuator responsive to a control circuit that is coupled to the cable adjacent a second end;
wherein the cable is selectively movable within the conduit according to movement of the cable actuator, and wherein the actuator is configured to adjust compression applied by the retention member according to movement of the cable relative to the conduit.

27. The automatic retention system of claim 26, wherein the cable actuator comprises
an electric motor mechanically coupled to a rotating member, the electric motor responsive to control input from the at least one control circuit;
wherein the at least one control circuit is programmed to control the electric motor to rotate in a first direction and a second direction opposite the first direction to adjust position of the cable relative to the conduit.

28. The automatic retention system of claim 26, wherein the conduit is anchored at a first end to a first cable mount of the at least one automatic retention apparatus, and wherein the conduit is anchored at a second end to a cable second mount of the cable actuator.

29. The automatic retention system of claim 26, comprising:
a garment configured to surround at least a portion of one of the one or more limbs, wherein the garment defines an interior passageway and the cable and conduit are positioned inside the interior passageway.

30. The automatic retention system of claim 1, comprising:
multiple cables separately positioned inside individual conduits, the multiple carafes extending between separate actuators of the multiple automatic retention apparatuses and the at least one control circuit;
multiple cable actuators responsive to the control circuit that are each individually coupled at a first end of each of the multiple cables;
wherein the multiple cables are selectively movable within the individual conduits according to movement of the cable actuators, and wherein the separate actuators are coupled to a second end of each of the multiple cables; and wherein the separate actuators are configured to adjust pressure applied by corresponding retention members according to movement of the individual cables relative to the individual conduits.

31. The automatic retention system of claim 1, wherein the actuator of at least one of the multiple automatic retention apparatuses comprises:
a rotating member with multiple teeth engaging one or more recesses defined by the retention member, wherein the rotating member is rotatable in a first direction to reduce tension of the retention member, and wherein the rotating member is rotatable in a second direction opposite the first direction to increase tension of the retention member;
wherein the actuator is mechanically coupled to the rotating member, wherein the actuator is arranged and configured to rotate the rotating member in the first direction and the second directions to increase or decrease tension of the retention member in response to input from the control circuit.

32. The automatic retention system of claim 31, wherein the retention member is an elongate retention member, wherein the one or more projections and recesses are defined in a portion of the retention member that is wider than it is thick, and narrower than it is long.

33. The automatic retention system of claim 31, wherein at least one of the one or more res and projections include through holes interspersed along the retention member.

34. The automatic retention system of claim 31, wherein the rotating ember rotates around an axis of rotation that is substantially parallel to a longitudinal axis defined by the retention member.

35. The automatic retention system of claim 31, wherein the rotating member rotates around an axis of rotation that is substantially perpendicular to a longitudinal axis defined by the retention member.

36. The automatic retention system of claim 31, wherein rotating the rotating member in a first direction displaces a first portion of the retention member relative to a second portion of the retention member to adjust tension of the retention member.

37. The automatic retention system of claim 1, wherein the actuator of at least one of the multiple automatic retention apparatuses comprises:
an electric motor mechanically coupled to the rotating member, the electric motor responsive to control input from the at least one control circuit, wherein the at least one control circuit is programmed to control the electric motor to rotate in the first and second direction to adjust tension of the retention member.

38. The automatic retention system of claim 1, wherein the actuator of at least one of the multiple automatic retention apparatuses comprises:
a rotating member engaging with at least a portion of the retention member, wherein the rotating member is rotatable in a first direction to reduce tension of the retention member, and wherein the rotating member is rotatable in a second direction opposite the first direction to increase tension of the retention member,
wherein the actuator is mechanically coupled to the rotating member, wherein the actuator is arranged and configured to rotate the rotating member in the first direction and the second directions to increase or decrease tension of the retention member in response to input from the control circuit.

39. The automatic retention system of claim 38, wherein the portion of the retention member that engages the rotating member is arranged and configured to wrap around the rotating member as it rotates.

40. The automatic retention system of claim 1, wherein a control circuit of the at least one control circuits is configured to control the actuators according to values for one or more operating parameters, and criteria for one or more rules.

41. The automatic retention system of claim 40, wherein the control circuit is configured to receive one or more values for the operating parameters from a remote computing device via a communications link.

42. The automatic retention system of claim 40, wherein the control circuit is configured to receive the criteria for the one or more rules from a remote computing device via a communications link.

43. The automatic retention system of claim 40, wherein the control circuit is configured to automatically determine and update at least one of the one or more values for one or more operating parameters and at least one of the criteria for the one or more rules.

44. The automatic retention system of claim 40, wherein the control circuit includes a memory, and wherein the control circuit is configured to retain an operational history of operating parameters including a first value of an operating parameter retained in the memory at a first time, and a second value of the operating parameter retained in the memory at a second later time, and wherein the first and second values are used to determine a third new value for the operating parameter.

45. The automatic retention system of claim 44, herein the operational history is sent to a remote computer via a communication link.

46. The automatic retention system of claim 1, wherein the actuator of at least one of the multiple automatic retention apparatuses comprises:
a manual control mechanically coupled to the rotating member, the manual control arranged and configured to rotate the rotating member in the first and second direction to adjust tension of the retention member based on input from a user.

47. The automatic retention system of claim 1, comprising:
one or more inflatable cavities positioned between the retention member and the at least one limb, wherein the actuator is arranged and configured to inflate the one or more inflatable cavities to adjust pressure applied to the at least one limb by the retention member.

48. An automatic retention system for applying tension to one or more limbs, comprising:
multiple automatic retention apparatuses that include:
a retention member surrounding a portion of at least one limb of the one or more limbs;
one or more inflatable cavities positioned between the retention member and the at least one limb; and
an actuator arranged and configured to inflate the one or more inflatable cavities to adjust pressure applied to the at least one limb by the retention member;
at least one sensor arranged and configured to sense changes in a sense parameter associated with the one or more limbs; and
at least one control circuit responsive to the at least one sensor, wherein the at least one control circuit is configured to control the actuators of the multiple retention apparatuses to adjust inflation of the one or more inflatable cavities according to input received from the at least one sensor.

49. The automatic retention system of claim 48, wherein the one or more inflatable cavities includes first and second cavities of the one or more inflatable cavities that are separate and distinct, and wherein the actuator is configured to selectively inflate the first cavity at a first pressure, and the second cavity at a second pressure that is different than the first pressure.

50. The automatic retention system of claim 48, wherein the one or more inflatable cavities are in fluid communication with each other.

51. The automatic retention system of claim 48, wherein the one or more inflatable cavities are arranged circumferentially around the retention member.

52. The automatic retention system of claim 48, comprising:
a fluid compression apparatus in fluid communication with one or more of the inflatable cavities;
wherein the fluid compression apparatus is arranged and configured to introduce a fluid into the cavities to inflate the one or more inflatable cavities.

53. The automatic retention system of claim 52, wherein the fluid compression apparatus is an air compressor responsive to the at least one control circuit, and wherein the fluid is air.

54. The automatic retention system of claim 52, wherein the fluid compression apparatus is a squeeze bulb, and wherein the fluid is air.

55. The automatic retention system of claim 52, comprising:
a one way valve configured to open when the compression apparatus introduces fluid into the cavities, and to otherwise automatically close to retain the fluid in the cavities.

56. The automatic retention system of claim 48, wherein the at least one sensor includes a first sensor mounted to a retention member of the multiple automatic retention apparatus that is configured to generate input based on an interior pressure of at least one of the one or more inflatable cavities, and a second sensor mounted to an object the retention member at least partially surrounds.
